(19) United States
(12) Patent Application Publication
(10) Pub. No.: US 2002/0150556 A1
(43) Pub. Date: Oct. 17, 2002

(54) Compositions and methods for tissue specific gene regulation therapy

(76) Inventors: Richard G. Vile, Rochester, MN (US); Kevin Harrington, London (GB); Steven Murphy, Sudbury (GB); Andrew Bateman, London (GB)

Correspondence Address: MARK S. ELLINGER, PH.D., Fish & Richardson P.C., P.A., Suite 3300, 60 South Sixth Street, Minneapolis, MN, 55402, US

(21) Appl. No.: 09/822,634

(22) Filed: Mar. 30, 2001

Related U.S. Application Data
(60) Provisional application No. 60/193,977, filed on Mar. 31, 2000.

(51) Int. Cl. A61K 48/00; C12N 15/86; C12N 5/08

(52) U.S. Cl. 424/93.2; 435/456; 435/366; 435/320.1

(57) ABSTRACT

This invention relates to a recombinant nucleic acid vector comprising a first expression cassette comprising a first promoter operably linked to a nucleic acid sequence encoding a syncytium-inducing polypeptide, wherein the first expression cassette is flanked on either side by a site recognized by a recombinase. The invention also includes a second expression cassette comprising a tissue-specific promoter operably linked to a nucleic acid sequence encoding a recombinase. The invention also includes cells and compositions including these expression cassettes and methods of reducing tumor volume by expression of these expression cassettes.

### RELATED APPLICATIONS

This application claims priority from U.S. Provisional Application No. 60/193,977 filed Mar. 31, 2000.

### BACKGROUND

Cell-cell fusion occurs naturally in some cell types, or in cells infected with any of a number of viruses encoding fusogenic proteins, or following chemical treatment of cells. Recruitment of cells into syncytia, large multinucleate agglomerations of fused cells, results in the death of the fused cells.

Fusogenic membrane glycoproteins (FMGs) have been found to induce syncytium formation when expressed in isolation from the remainder of the virus. International patent application No. WO98/40492 discloses a recombinant nucleic acid expression vector encoding a fusogenic membrane polypeptide from a virus, and a method of treating malignant disease, by administering to a patient the recombinant nucleic acid vector, where the vector is taken up by cancerous cells in the patient, causing the cancer cells to fuse and die.

There is a need in the art for improved methods of reducing the size of a tumor without causing damage to surrounding or adjacent tissue.

### SUMMARY

The invention encompasses a recombinant nucleic acid vector comprising a first expression cassette comprising a first promoter operably linked to a nucleic acid sequence encoding a syncytium-inducing polypeptide, wherein the first expression cassette is flanked on either side by a site recognized by a recombinase.

In one embodiment, the recombinant nucleic acid vector further comprises a second expression cassette comprising a tissue-specific promoter operably linked to a nucleic acid sequence encoding the recombinase.

In another embodiment, the first promoter is active in malignant cells, and the tissue specific promoter is active in non-malignant cells of the same lineage as the malignant cells but is substantially inactive in the malignant cells.

In another embodiment, the recombinase is selected from the group consisting of Cre recombinase, FLP recombinase, Gin recombinase, Pin recombinase, and lambda phage Integrase, and the site is susceptible to cleavage with the recombinase.

In another embodiment the first promoter is a tumor-specific promoter.

In another embodiment the tumor specific promoter is selected from the group consisting of a carcinoembryonic antigen (CEA) promoter, an alphafetoprotein promoter, a tyrosinase promoter, an Erb-B2 promoter and a myelin basic protein promoter.

In another embodiment, the sequence which encodes a syncytium-inducing polypeptide encodes a fusogenic membrane glycoprotein (FMG).

In another embodiment, the FMG is a viral FMG.

In another embodiment, the viral FMG is selected from the group consisting of type G membrane glycoprotein of rabies virus, type G membrane glycoprotein of Mokola virus, type G membrane glycoprotein of vesicular stomatitis virus, type G membrane glycoprotein of Togaviruses, murine hepatitis virus JHM surface projection protein, porcine respiratory coronavirus spike glycoprotein, porcine respiratory coronavirus membrane glycoprotein, avian infectious bronchitis spike glycoprotein and its precursor, bovine enteric coronavirus spike protein, paramyxovirus SV5 F protein, Measles virus F protein, canine distemper virus F protein, Newcastle disease virus F protein, human parainfluenza virus 3 F protein, simian virus 41 F protein, Sendai virus F protein, human respiratory syncytial virus F protein, Measles virus hemagglutinin, simian virus 41 hemagglutinin neuraminidase proteins, human parainfluenza virus type 3 hemagglutinin neuraminidase, Newcastle disease virus hemagglutinin neuraminidase, human herpesvirus 1 gH, simian varicella virus gH, human herpesvirus gB proteins, bovine herpesvirus gB proteins, cercopithecine herpesvirus gB proteins, Friend murine leukemia virus envelope glycoprotein, Mason Pfizer monkey virus envelope glycoprotein, HIV envelpoe glycoprotein, influenza virus hemaglutinin, poxvirus membrane glycoproteins, mumps virus hemaglutinin neuraminidase, mumps virus glycoproteins F1 and F2, West Nile virus membrane glycoprotein, herpes simplex virus membrane glycoprotein, Russian Far East encephalitis virus membrane glycoprotein, Venezuelan equine encephalitis virus membrane glycoproteinand varicella virus membrane glycoprotein.

In another embodiment, the vector is a retroviral vector.

The invention further encompasses a cell comprising a recombinant vector as described above.

The invention further encompasses a recombinant expression cassette system comprising a first expression cassette comprising a first promoter operably linked to a nucleic acid sequence encoding a syncytium-inducing polypeptide, wherein the first expression cassette is flanked on either side by a site recognized by a recombinase, and a second expression cassette comprising a tissue-specific promoter operably linked to a nucleic acid sequence encoding the recombinase.

In another embodiment, the first and second expression cassettes are encoded on a single vector nucleic acid.

In another embodiment, the first and second expression cassettes are encoded on separate nucleic acid vectors.

In another embodiment, the first promoter is active in malignant cells, and the tissue specific promoter is active in non-malignant cells of the same lineage as the malignant cells but is substantially inactive in the malignant cells.

In another embodiment, the recombinase is selected from the group consisting of Cre recombinase, FLP recombinase, Gin recombinase, Pin recombinase, and lambda phage Integrase, and the site is susceptible to cleavage with the recombinase.

In another embodiment, the first promoter is a tumor-specific promoter.

In another embodiment, the tumor specific promoter is selected from the group consisting of a carcinoembryonic antigen promoter, an alphafetoprotein promoter, a tyrosinase promoter, an Erb-B2 promoter and a myelin basic protein promoter.

In another embodiment, the sequence which encodes a syncytium-inducing polypeptide encodes an FMG.

In another embodiment, the FMG is a viral FMG.

In another embodiment, the viral FMG is selected from the group consisting of type G membrane glycoprotein of rabies virus, type G membrane glycoprotein of Mokola virus, type G membrane glycoprotein of vesicular stomatitis virus, type G membrane glycoprotein of Togaviruses, murine hepatitis virus JHM surface projection protein, porcine respiratory coronavirus spike glycoprotein, porcine respiratory coronavirus membrane glycoprotein, avian infectious bronchitis spike glycoprotein and its precursor, bovine enteric coronavirus spike protein, paramyxovirus SV5 F protein, Measles virus F protein, canine distemper virus F protein, Newcastle disease virus F protein, human parainfluenza virus 3 F protein, simian virus 41 F protein, Sendai virus F protein, human respiratory syncytial virus F protein, Measles virus hemagglutinin, simian virus 41 hemagglutinin neuraminidase proteins, human parainfluenza virus type 3 hemagglutinin neuraminidase, Newcastle disease virus hemagglutinin neuraminidase, human herpesvirus 1 gH, simian varicella virus gH, human herpesvirus gB proteins, bovine herpesvirus gB proteins, cercopithecine herpesvirus gB proteins, Friend murine leukemia virus envelope glycoprotein, Mason Pfizer monkey virus envelope glycoprotein, HIV envelpoe glycoprotein, influenza virus hemaglutinin, poxvirus membrane glycoproteins, mumps virus hemaglutinin neuraminidase, mumps virus glycoproteins F1 and F2, West Nile virus membrane glycoprotein, herpes simplex virus membrane glycoprotein, Russian Far East encephalitis virus membrane glycoprotein, Venezuelan equine encephalitis virus membrane glycoprotein, and varicella virus membrane glycoprotein.

In another embodiment, the expression cassette system is encoded on one or more retroviral vectors.

The invention further encompasses a cell comprising an expression cassette system as described above.

The invention further encompasses a therapeutic composition comprising a cell comprising an expression cassette system as described above, in admixture with a physiologically acceptable carrier.

The invention further encompasses a method of reducing tumor size, the method comprising the step of permitting expression in an individual in need of treatment for a disease caused by malignant cells of a first expression cassette comprising a first promoter operably linked to a nucleic acid sequence encoding a syncytium-inducing polypeptide, wherein the first expression cassette is flanked on either side by a site recognized by a recombinase, and a second expression cassette comprising a tissue-specific promoter operably linked to a nucleic acid sequence encoding the recombinase, wherein the first promoter is active in the malignant cells, and the tissue specific promoter is active in non-malignant cells of the same lineage as the malignant cells, but substantially inactive in the malignant cells, wherein the expression results in a reduction in tumor size.

In one embodiment, the step of permitting expression comprises the step of administering first and second expression cassettes to an individual in need of treatment for a disease caused by malignant cells.

In another embodiment, the recombinase is cre recombinase and said site recognized by a recombinase is a loxP site.

In another embodiment, the first promoter is a tumor-specific promoter.

In another embodiment, the tumor specific promoter is selected from the group consisting of a carcinoembryonic antigen promoter, an alphafetoprotein promoter, a tyrosinase promoter, an Erb-B2 promoter and a myelin basic protein promoter.

In another embodiment, the sequence which encodes a syncytium-inducing polypeptide encodes an FMG.

In another embodiment, the FMG is a viral FMG.

In another embodiment, the viral FMG is selected from the group consisting of type G membrane glycoprotein of rabies virus, type G membrane glycoprotein of Mokola virus, type G membrane glycoprotein of vesicular stomatitis virus, type G membrane glycoprotein of Togaviruses, murine hepatitis virus JHM surface projection protein, porcine respiratory coronavirus spike glycoprotein, porcine respiratory coronavirus membrane glycoprotein, avian infectious bronchitis spike glycoprotein and its precursor, bovine enteric coronavirus spike protein, paramyxovirus SV5 F protein, Measles virus F protein, canine distemper virus F protein, Newcastle disease virus F protein, human parainfluenza virus 3 F protein, simian virus 41 F protein, Sendai virus F protein, human respiratory syncytial virus F protein, Measles virus hemagglutinin, simian virus 41 hemagglutinin neuraminidase proteins, human parainfluenza virus type 3 hemagglutinin neuraminidase, Newcastle disease virus hemagglutinin neuraminidase, human herpesvirus 1 gH, simian varicella virus gH, human herpesvirus gB proteins, bovine herpesvirus gB proteins, cercopithecine herpesvirus gB proteins, Friend murine leukemia virus envelope glycoprotein, Mason Pfizer monkey virus envelope glycoprotein, HIV envelpoe glycoprotein, influenza virus hemaglutinin, poxvirus membrane glycoproteins, mumps virus hemaglutinin neuraminidase, mumps virus glycoproteins F1 and F2, West Nile virus membrane glycoprotein, herpes simplex virus membrane glycoprotein, Russian Far East encephalitis virus membrane glycoprotein, Venezuelan equine encephalitis virus membrane glycoprotein and varicella virus membrane glycoprotein.

In another embodiment, the step of administering comprises administering one or more retroviral vectors comprising the first and second expression cassettes.

In another embodiment, the step of administering comprises administering a cell comprising one or more recombinant nucleic acid vectors.

The invention further encompasses an expression cassette system comprising a first expression cassette comprising an hypoxic response element (HRE) operably linked to a nucleic acid sequence encoding a syncytium-inducing polypeptide, wherein the nucleic acid sequence encoding a syncytium-inducing polypeptide is flanked on either side by a sequence recognized by a recombinase, a second expression cassette comprising a tumor specific promoter operably linked to a nucleic acid sequence encoding a cytotoxic gene product, and a third expression cassette comprising a tumor specific promoter operably linked to the nucleic acid sequence encoding the recombinase.

The invention further encompasses an expression cassette system comprising a first expression cassette comprising an hypoxic response element (HRE) operably linked to a nucleic acid sequence encoding a syncytium-inducing polypeptide, wherein the nucleic acid sequence encoding a syncytium-inducing polypeptide is flanked on either side by sequences recognized by a recombinase, a second expression cassette comprising a tumor specific promoter operably linked to a nucleic acid sequence encoding a cytokine, and a third expression cassette comprising a tumor specific promoter operably linked to the nucleic acid sequence encoding the recombinase.

In one embodiment of either of the two preceding expression cassette systems, the vector is a retroviral vector.

In another embodiment, the tumor specific promoter is selected from the group consisting of a carcinoembryonic antigen promoter, an alphafetoprotein promoter, a tyrosinase promoter, an Erb-B2 promoter and a myelin basic protein promoter.

In another embodiment, the cytotoxic gene product is selected from the group consisting of HSV thymidine kinase, cytosine deaminase, nitroreductase, and a viral FMG.

In another embodiment, the cytokine is selected from the group consisting of IL-1, IL-2, IL-3, IL-4, IL-5, IL-6, IL-7, IL-12, GM-CSF, IFN-γ, and TNF-α.

The invention further encompasses a cell comprising either of the two preceding expression cassette systems.

In one embodiment, the cell is a macrophage.

The invention further encompasses a method of reducing the size of a tumor in an individual, the method comprising the step of permitting the expression in an individual of an expression cassette system comprising a first expression cassette comprising a nucleic acid sequence encoding a syncytium-inducing polypeptide, operably linked to an hypoxic response element (HRE), wherein the nucleic acid sequence encoding a syncytium-inducing polypeptide is flanked on either side by sequences recognized by a recombinase, a second expression cassette comprising a nucleic acid sequence encoding a cytotoxic gene product, operably linked to a tumor specific promoter, and a third expression cassette comprising a nucleic acid sequence encoding the, operably linked to the tumor specific promoter, wherein expression of the expression cassette system reduces the size of a tumor.

In one embodiment, the step of permitting expression comprises introducing the expression cassette system to a macrophage and introducing the macrophage to the individual.

The invention further encompasses a method of reducing the size of a tumor in an individual, the method comprising the step of permitting the expression in an individual of an expression cassette system comprising a first expression cassette comprising a nucleic acid sequence encoding a syncytium-inducing polypeptide, operably linked to an hypoxic response element (HRE), wherein the nucleic acid sequence encoding a syncytium-inducing polypeptide is flanked on either side by sequences recognized by a recombinase, a second expression cassette comprising a nucleic acid sequence encoding a cytokine, operably linked to a tumor specific promoter, and a third expression cassette comprising a nucleic acid sequence encoding the recombinase, operably linked to the tumor specific promoter, wherein expression of the expression cassette system reduces the size of a tumor.

In one embodiment, the step of permitting expression comprises introducing the expression cassette system to a macrophage and introducing the macrophage to the individual.

In another aspect, the invention features a macrophage-tumor cell hybrid. The hybrid can contain an expression cassette system containing a first expression cassette containing an hypoxic response element (HRE) operably linked to a nucleic acid sequence encoding a syncytium-inducing polypeptide, where the nucleic acid sequence encoding a syncytium-inducing polypeptide is flanked on either side by a sequence recognized by a recombinase, a second expression cassette containing a tumor specific promoter operably linked to a nucleic acid sequence encoding a cytotoxic gene product, and a third expression cassette containing a tumor specific promoter operably linked to the nucleic acid sequence encoding the recombinase. The hybrid can contain an expression cassette system containing a first expression cassette containing an hypoxic response element (HRE) operably linked to a nucleic acid sequence encoding a syncytium-inducing polypeptide, where the nucleic acid sequence encoding a syncytium-inducing polypeptide is flanked on either side by sequences recognized by a recombinase, a second expression cassette containing a tumor specific promoter operably linked to a nucleic acid sequence encoding a cytokine, and a third expression cassette containing a tumor specific promoter operably linked to the nucleic acid sequence encoding the recombinase.

Another aspect of the invention features a cell-tumor cell hybrid containing a hypoxic transcription factor. The hybrid can contain an expression cassette system containing a first expression cassette containing an hypoxic response element (HRE) operably linked to a nucleic acid sequence encoding a syncytium-inducing polypeptide, where the nucleic acid sequence encoding a syncytium-inducing polypeptide is flanked on either side by a sequence recognized by a recombinase, a second expression cassette containing a tumor specific promoter operably linked to a nucleic acid sequence encoding a cytotoxic gene product, and a third expression cassette containing a tumor specific promoter operably linked to the nucleic acid sequence encoding the recombinase. The hybrid can contain an expression cassette system containing a first expression cassette containing an hypoxic response element (HRE) operably linked to a nucleic acid sequence encoding a syncytium-inducing polypeptide, where the nucleic acid sequence encoding a syncytium-inducing polypeptide is flanked on either side by sequences recognized by a recombinase, a second expression cassette containing a tumor specific promoter operably linked to a nucleic acid sequence encoding a cytokine, and a third expression cassette containing a tumor specific promoter operably linked to the nucleic acid sequence encoding the recombinase.

Unless otherwise defined, all technical and scientific terms used herein have the same meaning as commonly understood by one of ordinary skill in the art to which the invention belongs. Although methods and materials similar or equivalent to those described herein can be used in the practice and testing of the present invention, suitable methods and materials are described. All publications, patent applications, patents and other references mentioned herein are incorporated by reference in their entirety. In case of conflict, the present specification, including definitions, will control. In addition, the materials, methods, and examples are illustrative only and are not intended to be limiting.

The details of one or more embodiments of the invention are set forth in the accompanying drawings and the description below. Other features, objects, and advantages of the invention will be apparent from the description and drawings, and from the claims.

As used herein, the term “recombinant nucleic acid vector” refers to a nucleic acid construct, generated by recombinant DNA methods, which is capable of being introduced into a cell, whereupon such construct directs the expression of one or more heterologous gene products within that cell.

As used herein, the term “expression cassette” refers to a nucleic acid sequence comprising a sequence encoding a polypeptide and an operably linked regulatory element sufficient to direct the transcription of the sequence encoding the polypeptide. As used herein, the term “operably linked” means that the two sequences are joined such that the regulatory element is placed in a position and orientation such that expression of the joined coding sequence occurs under the direction of that regulatory element. An expression cassette may comprise a simple or basal promoter, or a promoter plus enhancer and/or silencer combination. Further, a given expression cassette may direct regulated or constitutive expression of the linked coding sequence. An expression cassette contains recombinant DNA and is found on an extracxhromosomal element, such as a plasmid or an episome, which may become integrated into a chromosome.

As used herein, the term “regulatory element” refers to those sequences necessary and sufficient to direct the transcription of a linked nucleic acid sequence as required for a given application. For example, a minimally active basal promoter may be required or desired in some applications, while a highly active or tissue-specific promoter plus an enhancer may be required or desired for others. The term “regulatory element” is meant to encompass the full range of such situations.

As used herein, the term “expression cassette system” refers to one or more expression cassettes. Thus, a system may be one, two, three, etc., plasmids or one, two three, etc., episomes, which may become integrated into a genome; or two or more cassettes may be contained on a single piece of DNA.

As used herein, the term “tumor specific” refers to a property that is characteristic of tumor cells. By “property” is meant the presence of an antigen or group of antigens or polypeptide markers expressed within or on a tumor cell, expression of a particular gene or group of genes by a tumor cell, a function of a tumor cell (e.g., invasion of tissues, production of a growth factor or stimulation of angiogenesis), or a particular morphology. In the present application, “tumor specific” is used in reference to a gene regulatory element (promoter or promoter plus enhancer and/or silencer), the gene it encodes, or the polypeptide product of such a gene. In the context of a gene regulatory element or a “tumor specific promoter”, the term means that the promoter directs the transcription of a linked sequence in a tumor cell, but is substantially inactive in a fully differentiated non-tumor cell of the same lineage. It is to be understood that a basal or minimal promoter element from a given gene may be active on an operably linked heterologous sequence, but that such a basal or minimal promoter does not necessarily confer tumor-specific expression on such a sequence. Rather, tumor-specific expression may further require sequences, such as upstream or downstream enhancer or even silencer elements, in addition to the basal promoter sequences to drive the tumor-specific expression of linked sequences. The term “tumor-specific promoter”, as used herein, is meant to encompass any such upstream or downstream sequences required to provide tumor-specific transcription of an operably linked nucleic acid sequence. A tumor-specific promoter according to the invention is at least 1,000 times more active in an appropriate tumor cell, in terms of the amount of transcription directed by the promoter, than in a non-fetal, non-tumor cell of the same lineage.

When used in the context of a gene or the polypeptide product encoded by a gene, the term “tumor-specific” means that the product of the gene is detectable in or on one or more tumor cell types, but is not detected in or on non-fetal, non-tumor cells of the same lineage(s).

As used herein, the terms “substantially inactive” or “substantial lack of activity”, when used to refer to a promoter means that the polypeptide product of a gene linked to a particular promoter is not detectable in or on a given cell or tissue. Detection of a polypeptide product may be based, for example, on binding of an antibody, or it may be based on measurement of an activity of the polypeptide product, such as an enzyme activity or a ligand binding activity.

As used herein, the term “substantially active,” when used to refer to a promoter means that the polypeptide product of a gene linked to a particular promoter is detectable in a given cell or tissue. Detection of a polypeptide product may be based, for example, on binding of an antibody, or it may be based on measurement of an activity of the polypeptide product, for example an enzyme activity, a ligand binding activity or.

As used herein, the term “active promoter” means that the promoter directs transcription of linked sequences that is detectable at the RNA level, the polypeptide level, or both. An active promoter, as used herein, produces a hybridization signal that is at least 5 fold higher than background in a nuclear run-on transcription assay. The nuclear run-on transcription assay allows the measurement of initiated transcription activity of particular genes in isolated nuclei by allowing the extension of transcripts initiated in vivo to continue in vitro in the presence of one or more labeled ribonucleotides. The labeled nucleotides are isolated and then hybridized to immobilized probes specific for the genes of interest (in this case, the gene driven by a promoter of interest). In the assay, background signal is determined by the amount of hybridization signal detected on a probe, such as a plasmid or bacteriophage, that has no corresponding sequence in the genome of the species (e.g., human) from which the nuclei are isolated. The nuclear run-on transcription method is well known to those skilled in the art, and is described by, for example, Ausubel et al. (1988, Current Protocols in Molecular Biology, John Wiley & Sons, Inc.).

As used herein, the term “tissue specific” refers to a characteristic of a particular tissue that is not generally found in all tissues, or may be exclusive found in a tissue of interest. In the present application, “tissue specific” is used in reference to a gene regulatory element (promoter or promoter plus enhancer and/or silencer), the gene it encodes, or the polypeptide product of such a gene. In the context of a gene regulatory element or a “tissue specific promoter”, the term means that the promoter (and also other regulatory elements such as enhancer and/or silencer elements) directs the transcription of a linked sequence in a non-fetal cell of a particular lineage, tissue, or cell type, but is substantially inactive in cells or tissues not of that lineage, tissue, or cell type. A tissue-specific promoter useful according to the invention is at least 5-fold, 10-fold, 25-fold, 50-fold, 100-fold, 500-fold or even 1,000 times more active in terms of transcript production in the particular tissue than it is in cells of other tissues or in transformed or malignant cells of the same lineage. In the context of a gene or the polypeptide product of a gene, the term tissue specific means that the polypeptide product of the gene is detectable in cells of that particular tissue or cell type, but not substantially detectable in certain other cell types.

“Detectable” means that an RNA product of a gene is identifiable in cells in which it is produced (that is, cells for which it is tissue specific) at a level which is at least 2-fold, preferably, 5-fold, 10-fold, 50-fold or higher, relative to cells in which the RNA product is not substantially detectable; for example, in an RT PCR assay, an RNA may be detectable in cells of a tissue of interest (tissue specific) at a level which is at least 10 fm, or 50 fm, 100 fm, 500 fm or higher; in an RNA dot blot assay, an RNA may be detectable in tissue specific cells at a level which is determined by scanning densitometry of an autoradiogram of the blot to be at least 2-fold or higher than in cells that are not of that tissue type. Generally, RNA that is detectable will be present at a level which is determined to be at least 50 ng or higher, such as 100 ng, 400 ng, 500 ng, or higher, whereas “not substantially detectable” refers to less than 40 ng, such as 25 ng, 10 ng, 5 ng, or undetectable.

“Detectable” also may be used with respect to a polypeptide or a fragment there of is identifiable in cells in which it is produced (that is, cells for which it is tissue specific) at a level which is at least 2-fold, preferably, 5-fold, 10-fold, 50-fold or higher, relative to cells in which the polypeptide is not substantially detectable; for example, in an immunoprecipitation assay, a polypeptide may be detectable in tissue specific cells at a level which is determined to be at least 2-fold or higher than in cells that are not of that tissue type. Generally, a polypeptide that is detectable will be present at a level which is determined to be at least 5 ng or higher, such as 10 ng, 40 ng, 50 ng, or higher, whereas “not substantially detectable” refers to less than 4 ng, such as 2.5 ng, 1.0 ng, 0.5 ng, or undetectable.

As used herein, the term “malignant cell” refers to a cell that is oncogenically transformed. Characteristics of malignant cells include anomalous behavior in tissue culture (for example, growth factor independence, loss of contact inhibition, capacity for anchorage-independent growth, growth to higher density than non-tumor cells, and failure to reach senescence after multiple passages), the ability to invade tissues or metastasize to distant sites, the ability to form tumors when injected into nude mice, and the ability to stimulate anglogenesis.

As used herein, the phrase “tumor specific promoter is active in malignant cells” means that a given promoter directs the transcription of a linked sequence in oncogenically transformed or malignant cells of a particular type. Further, the phrase means that the polypeptide product of the linked sequence is detectable in transformed or malignant cells.

As used herein, the terms “non-malignant cell”, “non-transformed cell” and “non-tumor cell” refer to a cell that is not oncogenically transformed. A non-malignant, non-transformed or non-tumor cell cannot form tumors in nude mice, nor can it grow indefinitely in culture or proliferate in semisolid medium. A non-malignant cell is contact-inhibited when placed in tissue culture.

As used herein, the phrase “tissue specific promoter is active in non-malignant cells” means that a given promoter directs the transcription of a linked sequence in non-transformed or non-malignant cells of a given tissue or cell type. The phrase also means that the polypeptide product of the linked sequence is detectable in cells of a particular non-transformed or non-malignant tissue or cell type.

As used herein, the terms “syncytium” or “syncytia” refer to multinucleate agglomerations of cells formed by fusion of their membranes.

As used herein, the term “syncytium-inducing polypeptide” refers to a membrane polypeptide or a portion thereof that causes cell fusion, with such cell fusion leading to the formation of syncytia. Syncytium-inducing polypeptides according to the invention encompass those proteins naturally produced by viruses, particularly the so-called fusogenic membrane proteins (FMPs) and fusogenic membrane glycoproteins (FMGs), that mediate virus-cell fusion, as well as cell-cell fusion of infected cells. Syncytium-inducing polypeptides according to the invention further encompass non-viral polypeptides known to mediate cell-cell fusion events in vivo. A “viral fusogenic membrane glycoprotein” is a virally-derived fusogenic membrane protein that, in nature, mediates membrane fusion of a virus to its host target cell. A syncytium-inducing polypeptide (or portion thereof) or fusogenic membrane glycoprotein (or portion thereof), as used herein, has the ability, when in isolation from a virus, to mediate or induce fusion between a cell expressing the fusogenic membrane glycoprotein and a cell expressing a receptor for the fusogenic membrane glycoprotein. Examples of fusogenic membrane proteins include, but are not limited to fertilin b. The viral fusogenic membrane glycoprotein subset of the fusogenic membrane proteins includes, but is not limited to: type G glycoproteins in Rabies, Mokola, vesicular stomatitis and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus, canine distemper virus, Newcastle disease virus, human parainfluenza virus 3, simian virus 41, Sendai virus and human respiratory syncytial virus; gH of human herpesvirus 1 and simian varicella virus, with the charepone protein gL; human, bovine and cercopithicine herpesvirus gB; envelope glycoproteins of Friend murine leukemia virus and Mason Pfizer monkey virus; influenza haemagglutinin; G protein of Vesicular Stomatitis Virus; mumps virus hemagglutinin neuraminidase, and glycoproteins F1 and F2; and membrane glycoproteins from Venezuelan equine encephalomyelitis.

It is recognized herein that some syncytium-inducing polypeptides function alone, while others require more than one different polypeptide to have fusion-promoting activity. As used herein then, the singular term “syncytium-inducing polypeptide” is meant to encompass single fusion-promoting polypeptides as well as each of the polypeptides required for promoting fusion when there is a requirement for more than one.

As used herein, the term “recombinase” refers to an enzyme which catalyzes the exchange or excision of DNA segments at specific recombination sites. The recombination sites for a given recombinase are specific DNA sequences recognized by that recombinase, and for each recombinase useful according to the invention there is a single recombination site sequence that must flank the sequence to be excised. Therefore, while different recombinases useful according to the invention have different recombination site sequences, each recombinase has a particular corresponding recombination site sequence that is unique to that recombinase, and is in fact required for the excision function of that recombinase.

As used herein, the term “flanked by sites recognized by a recombinase” means that a selected sequence has a recognition site sequence for a recombinase situated on both sides of that selected sequence. Recombination in vivo may occur between recombinase recognition site sequences separated by many kilobases of DNA sequence. However, it is preferred herein that the recombinase recognition site sequences are located within 50 to 1,000 base pairs 5′ or 3′, respectively, from the 5′ and 3′ ends of the expression cassette. As noted elsewhere herein, the recombinase recognition site sequences may flank simply the coding sequence, or even part of the coding sequence one wishes to excise or they may flank the entire expression cassette. It is understood that the recombinase recognition site sequences on either side of the selected sequence are oriented as direct repeats such that excision of the selected sequence between them occurs in the presence of the corresponding recombinase that recognizes those sites. While it is preferred that both recognition site sequences are identical, mutations that alter one site relative to another or both sites relative to a wild-type recombinase recognition sequence are tolerated by some recombinases. Constructs or expression cassettes bearing mutated recombinase recognition sites are useful in the methods of the invention if the mutated sites can serve as substrates for recombinase-mediated excision. Assays for recombinase-mediated excision are known in the art. See, for example, Abremski et al., 1983, Cell 32: 1301-1311; Sauer et al., 1989, Nucl. Acids Res. 17: 147-161; and Sauer et al., 1988, Proc. Natl. Acad. Sci. U.S.A. 85: 5166-5170.

As used herein, the term “retroviral vector” refers to a recombinant nucleic acid vector that is derived from or based upon a retrovirus. According to the invention, a retroviral vector has the virally-derived coding sequences necessary, at least when introduced to an appropriate cell line (i.e., a packaging cell line) to produce viral particles capable of infecting at least one cell type. A retroviral vector according to the invention is capable of carrying and delivering exogenous expression cassettes necessary for the methods of the invention.

As used herein, the term “hypoxic response element” or “HRE” refers to a gene regulatory element that confers hypoxia-sensitive expression upon sequences operably linked to it. As used herein, hypoxia-sensitive expression means that the regulatory element is transcriptionally active when a cell containing such an element is exposed to hypoxic conditions or is in a state of hypoxia. As used herein, “hypoxic conditions” mean that the concentration of oxygen available in a particular environment or microenvironment is low enough to activate expression of an HRE-linked gene construct. An HRE is described by Lok & Ponka, 1999, J. Biol. Chem. 274:24147-24152.

As used herein, the term “cytotoxic gene product” refers to a polypeptide that causes the death of a cell that expresses it.

As used herein, the term “macrophage” refers to a phagocytic cell of the monocyte lineage that occurs within most normal tissues (so-called “resident macrophages”). Resident macrophages can be activated by cytokines and other stimuli to produce a wide variety of biologically active products, for example, enzymes, such as proteases, phosphatases and lipases, complement components, coagulation factors, reactive oxygen intermediates, eicosanoids, cytokines, growth factors and nitric oxide. Macrophages useful according to the invention express at least the following combination of cell surface markers: CD11a, -b, and -c, CD16, CD17, CD63, CD64, CD68 and CD71.

As used herein, the term “hypoxic environment surrounding malignant cells” refers to the oxygen-poor microenvironment near a tumor. As used herein, the term refers to an area with an oxygen concentration that is sufficiently low to activate expression of a hypoxic response element-containing gene construct. “Activated expression”, when used in reference to an HRE-linked construct means at least a 10-fold increase in the amount of transcripts detectable from the construct relative to the expression in cells not exposed to an hypoxic environment. Alternatively, “activated expression” means that the polypeptide product encoded by the HRE-linked gene construct is detectable, for example, through immunochemical or enzymatic functional assays.

As used herein, the term “cytokine” refers to a protein that stimulates an immune response in a patient, including but not limited to IL-1, IL-2, IL-3, IL-4, IL-5, IL-6, IL-7, IL-12, GM-CSF, IFN-γ and TNF-α or any other protein that stimulates an immune response. A protein or polypeptide antigen, i.e., a protein or polypeptide that elicits an immunoglobulin response specific to that protein or polypeptide is specifically excluded from the meaning of the term “cytokine” as used herein.

### DESCRIPTION OF THE DRAWINGS

FIG. 1 shows a schematic representation of the mechanism of the first aspect of the invention directed to reducing the size of a tumor while essentially limiting damage to adjacent non-tumor tissue.

FIG. 2 shows a schematic representation of the mechanism of the second aspect of the invention directed to reducing the size of a tumor while essentially limiting damage to adjacent non-tumor tissue.

FIG. 3 shows the genomic nucleotide sequence of the human CEA gene, including the promoter (SEQ ID NO: 1).

FIG. 4 shows the sequence of the melanoma-specific human tyrosinase promoter from −300 to −1, relative to the transcription start site (SEQ ID NO: 2).

FIG. 5 shows the results of experiments evaluating the effect on syncytium formation when cells expressing a LoxP-flanked fusogenic membrane protein gene sequence (“Tel”) are mixed with cells expressing Cre recombinase (293-Cre). FIG. 5A shows the results of varying the ratio of Tel cells and 293-Cre cells on syncytial killing. FIG. 5B shows an agarose gel after separation of Hirt DNA supernatants from mixtures of cells Tel and 293-Cre cells at the ratios shown in FIG. 5A.

FIG. 6 shows the results of experiments demonstrating the transfer and activity of tumor-specific transcription factors from tumor cells to non-tumor cells. HT1080 cells stably transfected with an IL-2 gene operably linked to a tumor-specific tyrosinase promoter were mixed with cells from six different cell lines either expressing (“/GALV”, odd numbered columns) or not expressing (even numbered columns) a GALV FMG polypeptide. Secretion of IL-2 from the mixed cultures for each mixture is represented on the Y axis.

FIG. 7 shows the constructs used in transient transfection experiments. The three following constructs are shown; pCR3.1-GALV, GALC-ON and GALV-OFF.

FIG. 8 shows a schematic representation of the first primer pair used in diagnostic PCR of Hirt DNA extracts.

FIG. 9 shows a schematic representation of the second primer pair used in diagnostic PCR of Hirt DNA extracts.

FIG. 10 shows RT-PCR analysis of HCT-116 cells transfected with pCR3.1-GALV and GALV-OFF. RT-PCR for GALV message shows the activity of the ON and OFF switches in cre +ve and cre −ve cell lines. In the upper panel, primers for GALV were used. In the lower primers for GAPDH were used. Lane assignments are as follows: L=1 kbp ladder; Lane 1=HCT-116 cells transfected with pCR3.1-GALV, RT +ve; Lane 2=HCT-116 cells transfected with GALV-ON, RT +ve; Lane 3=HCT-116 cells transfected with GALV-OFF, RT +ve; Lane 4=HCT-116 cells transfected with pCR3.1-GALV, RT −ve; Lane 5=HCT-116 cells transfected with GALV-ON, RT −ve; Lane 6=HCT-116 cells transfected with GALV-OFF, RT −ve; Lane 7=293-crc cells transfected with pCR3.1-GALV, RT +ve; Lane 8=293-cre cells transfected with GALV-ON, RT +ve; Lane 9=293-cre cells transfected with GALV-OFF, RT +ve; Lane 10=293-cre cells transfected with pCR3.1-GALV, RT −ve; Lane 11=293-cre cells transfected with GALV-ON, RT −ve; Lane 12=293-cre cells transfected with GALV-OFF, RT −ve; Lane 13=PCR water control; and Lane 14=PCR cDNA +ve control.

FIG. 11 shows PCR results of Hirt extracted DNA, confirming the activity of the OFF switch in cre +ve but not cre −ve cell lines. The first primer pair was used. Lane assignments are as follows: L1=100 bp ladder; Lane 1=Ha-116 cells transfected with pCR3.1-GALV; Lane 2=HCT-116 cells transfected with GALV-ON; Lane 3=HCT-116 cells transfected with GALV-OFF; Lane 4=293-cre cells transfected with pCR3.1-GALV; Lane 5=293-a cells transfected with GALV-ON; Lane 6=293-cre cells rransfected with GALV-OFF; Lane 7=B lank; Lane 8=pCR3.1-GALV cDNA +ve; Lane 9=GALV-ON cDNA +ve; Lane 10=GALV-OFF cDNA +ve; and L2=1 kbp ladder.

FIG. 12 shows the results of PCR analysis of Hirt extracted DNA in GALV transfected HCT-116 cells fused with heterologous cre +ve or cre −ve cell lines. The first primer pair was used in reactions 1-7, which will amplify either a 130 bp or 2.5 kbp band depending upon the activity of cre recombinase on the loxP sites. THE second primer pair was used for reactions 9-15, which will amplify either no band or a 800 bp band depending upon the activity of cre recombinase on the loxP sites. The lane assignments are as follows: L1=1 kbp ladder; L2=100 bp ladder; Lane 1=HCT-116 transfected with GALV-OFF mixed with HCT-116; Lane 2=HCI′-116 transfected with GALV-OFF mixed with HuH7; Lane 3=HCT-116 transfected with GALV-OPF mixed with 293-cre; Lane 4=HCT-116 transfected with GALV-OFF mixed with HuH7-cre15; Lane 5=HCT-116 transfected with GALV-OFF mixed with HuH7-cre21; Lane 6=PCR GALV-OFF cDNA +ve control; Lane 7=PCR water control; Lane 8=HCT-116 transfected with GALV-OFF mixed with HCT-116; Lane 9=HCT-116 transfected with GALV-OFF mixed with HuH7; Lane 10=HCT-116 transfectcd with GALV-OFF mixed with 293-cre; Lane 11=HCT-116 transfected with GALV-OFF mixed with HuH7-crel 5; Lane 12=HCT-116 transfected with GALV-OFF mixed with HuH7-cre21; Lane 13=PCR GALV-OFF cDNA +ve control; and Lane 14=PCR water control.

FIG. 13 shows additional constructs in which FMG cassettes flanked by loxP sites are under the control of tissue specific promoters.

### DETAILED DESCRIPTION

Mechanism of Action

In a first aspect, the invention relates to a method of reducing the size of a tumor or reducing the number of malignant cells (i.e., reducing malignant or tumor cell load) while essentially sparing surrounding, non-tumor or non-malignant tissues from damage. In its broadest sense, the invention relates to a method of reducing the size of a tumor or reducing tumor cell load by introducing a first nucleic acid expression cassette to the vicinity of a tumor, wherein the nucleic acid expression cassette carries a nucleic acid sequence encoding a promoter operably linked to a nucleic acid sequence encoding a syncytium-inducing polypeptide. The first expression cassette, or simply the nucleic acid sequence encoding the syncytium-inducing polypeptide, is flanked on either side by a site that is recognized and cleavable by a recombinase. In this aspect of the invention, a second expression cassette comprising a tissue specific promoter operably linked to a nucleic acid sequence encoding the recombinase that recognizes the sites flanking the first expression cassette or its polypeptide coding sequence is also introduced to the vicinity of the tumor. The cassette may be encoded by one vector or two separate vectors and may be introduced simultaneously or at different times.

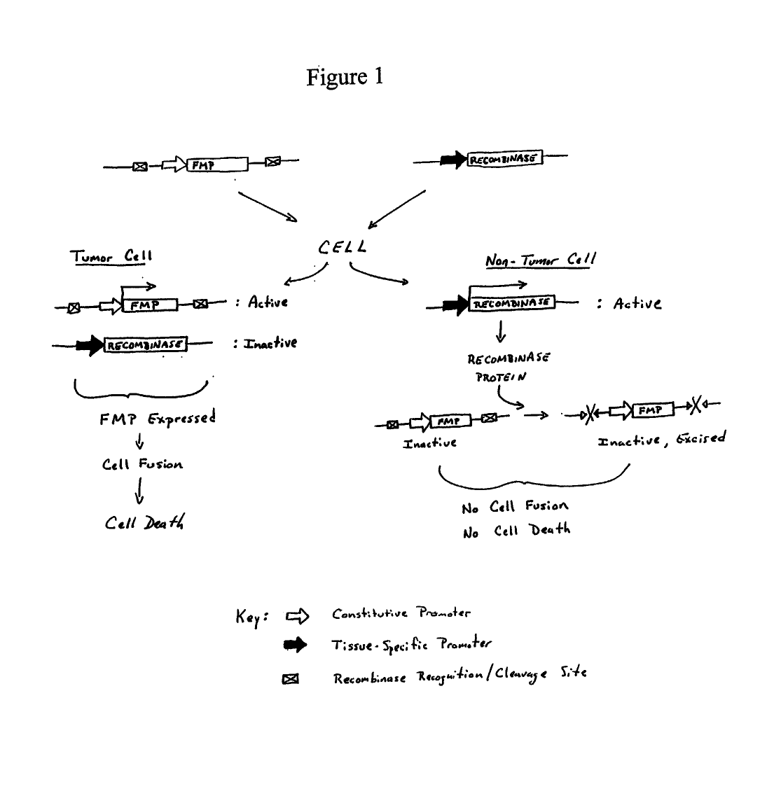

The result of the introduction of a vector comprising such first and second expression cassettes to a cell varies depending upon the activity of the tissue specific promoter in that cell. While not wishing to be bound by any specific theory, the mechanism of action believed by the inventors to function in the methods of the first aspect of the invention is schematically diagrammed in FIG. 1. It is believed, for example, that when the cassettes are introduced to a tumor cell in which the promoter driving the syncytium-inducing polypeptide is substantially active and the tissue specific promoter is substantially inactive, the activation of the first expression cassette results in expression and display of the syncytium-inducing polypeptide on the surface of the cell. In such a case, when the syncytium-inducing polypeptide contacts an appropriate receptor on an adjacent cell, cell fusion occurs. Continued expression of the syncytium-inducing polypeptide on the cell surface of the fused cells is believed to result in recruitment of additional cell fusion partners, leading to formation of a non-viable syncytium.

In contrast, when the expression cassettes are introduced to a cell in which the tissue specific promoter driving the expression of the recombinase cassette is active, it is believed that the expression of the recombinase results in excision of the expression unit for the syncytium-inducing polypeptide. This excision is believed to inactivate the expression of the syncytium-inducing polypeptide, thereby inactivating the fusion capacity of the cell containing the construct.

Because tissue specific promoters are normally highly expressed in non-transformed cells, including those cells immediately adjacent to a mass of tumor cells, the recombinase linked to an appropriate tissue-specific promoter will assure the excision of the syncytium-inducing expression cassette from the vector if it is introduced to a non-tumor cell. Similarly, when a tumor cell fuses with an adjacent non-tumor cell through expression of a syncytium-inducing polypeptide encoded by a vector of the invention, the tissue-specific transcription factors in the non-tumor cell activate the recombinase expression cassette. This activation induces excision of the syncytium-inducing cassette. This excision occurring in non-tumor cells and in tumor cells at the tumor/non-tumor interface or margin serves to limit the damage to non-tumor tissues surrounding or adjacent to a tumor, while having no effect on the continued expression of the syncytium-inducing polypeptide in tumor cells that are not adjacent to the margin.

The promoter drivng the expression of the syncytium-inducing polypeptide may be any constitutive promoter (e.g., the CMV promoter), but may optionally be a tumor-specific promoter. The use of a tumor-specific promoter will further limit the degree to which the syncytium-inducing polypeptide is expressed in non-tumor tissues.

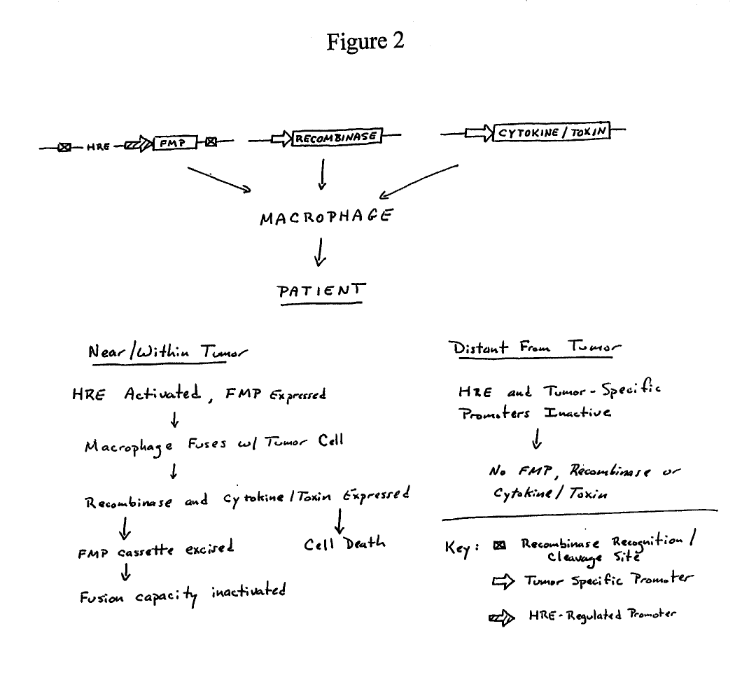

In a second aspect, the invention relates to methods of reducing the size of a tumor in an individual comprising introducing a first expression cassette comprising an hypoxic response element (HRE)-regulated promoter operably linked to a nucleic acid sequence encoding a syncytium-inducing polypeptide, in which the whole first expression cassette or simply the sequence encoding the syncytium-inducing polypeptide is flanked by sites recognized by a recombinase. In this aspect of the invention, a second expression cassette is introduced, comprising a tumor-specific promoter operably linked to a nucleic acid sequence encoding a cytokine or a cytotoxic gene product, and a third expression cassette is introduced, comprising a tumor-specific promoter operably linked to a nucleic acid sequence encoding the recombinase that recognizes the sites flanking the first expression cassette. When each of these expression cassettes is introduced to a macrophage and the macrophage is administered to an individual, it is believed that the expression cassettes will be substantially inactive until the macrophage migrates to the vicinity of a tumor or tumor cell syncytium (Diagrammed in FIG. 2). Once in the relatively hypoxic environment common to tumors, activation of the HRE commences expression of the syncytium-inducing polypeptide, which causes fusion of the macrophage with adjacent tumor cells. The fusion will introduce the construct to the tumor cell, wherein one or more tumor specific promoters are active. The expression cassettes encoding the cytotoxic gene product or cytokine and the recombinase are then activated. Expression of a cytotoxic gene product results in death of cells expressing it, thereby reducing tumor size. Alternatively, expression of a cytokine increases the anti-tumor immune cell activity, also resulting in a reduction in tumor size. The activation of the tumor specific promoter driving the recombinase gene sequence is believed to result in a limitation of the damage to surrounding cells caused by this treatment method. That is, the activation of the recombinase cassette caused by fusion of the carrier macrophage with a tumor cell results in inactivation of the syncytium-inducing ability of the fused cell, yet the cytotoxic gene or cytokine gene remains fully activated. As in the first aspect of the invention, the cassettes may be located on one or more recombinant nucleic acid vectors, and the vectors comprising the cassettes may be administered simultaneously or at different times. Also, the tumor-specific promoters on the respective expresion cassettes may be the same or different.

It is contemplated that the second aspect of the invention, while useful for reducing tumor size when used alone, may be used to advantage in conjunction with the method of the first aspect of the invention or with any method that induces tumor cell syncytia. The simultaneous use of a cassette encoding a syncytium-inducing polypeptide, a cytokine or a cytotoxic product and a recombinase with a cassette encoding a syncytium-inducing polypeptide and a recombinase as described above in the second and first aspects of the invention has the advantage of amplifying the anti-tumor effect of the syncytia-inducing treatment approach.

Methods According to the Invention

The methods of the invention make use of a number of components and bodies of information known in the art. For example, the invention makes use of syncytium-inducing polypeptides, tumor- and tissue-specific promoters, cytotoxic gene products, cytokines, recombination systems, and nucleic acid vectors and their introduction to cells. The characteristics of those components necessary to the practice of the invention are described in detail below.

A. Syncytium-inducing Polypeptides According to the Invention.

Syncytium formation is the result of cell-cell fusion events. Cell-cell fusion is induced by causing one of the cells or cell types intended to undergo fusion to express any of a series of syncytium-inducing polypeptides or fusogenic membrane polypeptides (FMPs). Cells expressing one or more FMPs serve as fusion donor partners with acceptor target cells. A large number of FMPs are known to those skilled in the art, including FMPs expressed by viruses and by various cell types that naturally undergo cell fusion.

1. Virally-derived FMPs.

One large family of FMPs is that comprising FMPs expressed by viruses. Many viruses depend upon fusogenic membrane glycoproteins (which constitute a subset of FMPs) displayed upon their outer surfaces in order to fuse with and enter target cells. These proteins frequently function to induce cell-cell fusion when expressed in isolation from the remainder of the viral genes. Viral fusogenic polypeptide FMGs, both naturally occurring and engineered by recombinant nucleic acid techniques, and suitable for use in the present invention are described in detail in WO 98/40492, the content of which is incorporated herein by reference.

Viral syncytium-inducing polypeptides useful according to the invention include fusogenic membrane glycoproteins which include but are not limited to the following.

a) Membrane Glycoproteins of Enveloped Viruses.

Enveloped viruses have membrane spike glycoproteins for attachment to mammalian cell surfaces and for subsequent triggering of membrane fusion, providing a pathway for viral entry into the cell. In some viruses, attachment and fusion triggering are mediated by a single viral membrane glycoprotein, but in others these functions are provided by two or more separate glycoproteins. Sometimes (e.g. Myxoviridae, Togaviridae, Rhabdoviridae) the fusion triggering mechanism is activated only after the virus has entered into the target cell by endocytosis, at acid pH (i.e., below about pH 6.0). Examples of such membrane glycoproteins in Rhabdoviruses are those of type Gin rabies (Genbank Acc. No. U11736), Mokola (Genbank Ace. No. U17064) and vesicular stomatitis (Genbank Acc. Nos. M21417 and J04326) viruses, and those in Togaviruses.

Other viruses (e.g. Paramyxoviridae, Retroviridae, Herpesviridae, Coronaviridae) can fuse directly with the target cell membrane at substantially neutral pH (about 6.0-8.0) and have an associated tendency to trigger membrane fusion between infected target cells and neighboring noninfected cells. The visible outcome of this latter tendency for triggering of cell-cell fusion is the formation of cell syncytia containing up to 100 nuclei. Viral membrane proteins of these latter groups of viruses are of particular interest in the present invention. In addition to those proteins from Paramyxoviruses, Retroviruses and Herpesviruses discussed below, examples of Coronavirus membrane glycoprotein genes include those encoding the murine hepatitis virus JHM surface projection protein (Genbank Ace. Nos. X04797, D00093 and M34437), porcine respiratory coronavirus spike- and membrane glycoproteins (Genbank Acc. No. Z24675) avian infectious bronchitis spike glycoprotein (Genbank Acc. No. X64737) and its precursor (Genbank Acc. No. X02342), and bovine enteric coronavirus spike protein (Genbank Acc. No. D00731).

b) Viral Membrane Glycoproteins of the Paramyxoviridae Viruses.

Viruses of the Family Paramyxoviridae have a strong tendency for syncytium induction which is dependent in most cases upon the co-expression of two homo-oligomeric viral membrane glycoproteins, the fusion protein (F) and the viral attachment protein (H, HN or G). Co-expression of these paired membrane glycoproteins in cultured cell lines is required for syncytium induction although there are exceptions to this rule such as SV5 whose F protein alone is sufficient for syncytium induction. F proteins are synthesized initially as polyprotein precursors (F₀) which cannot trigger membrane fusion until they have undergone a cleavage activation. The activating protease cleaves the F₀ precursor into an extraviral F₁ domain and a membrane-anchored F₂ domain which remain covalently associated through disulfide linkage. The activating protease is usually a serine protease and cleavage activation is usually mediated by an intracellular protease in the Golgi compartment during protein transport to the cell surface. Alternatively, where the cleavage signal is not recognized by a Golgi protease, the cleavage activation can be mediated after virus budding has occurred, by a secreted protease (e.g. trypsin or plasmin) in an extracellular location (Ward et al. Virology, 1995, 209, p 242-249; Paterson et al., J. Virol., 1989, 63, 1293-1301).

Examples of Paramyxovirus F genes include those of Measles virus (Genbank Acc. Nos. X05597 or D00090), canine distemper virus (Genbank Acc. No. M21849), Newcastle disease virus (Genbank Acc. No. M21881), human parainfluenza virus 3 (Genbank Acc. Nos. X05303 and D00125), simian virus 41 (Genbank Acc. Nos. X64275 and S46730), Sendai virus (Genbank Acc. No. D11446) and human respiratory syncytial virus (Genbank Acc. No. M11486, which also includes glycoprotein G). Also of interest are Measles virus hemagglutinin (Genbank Acc. No. M81895) and the hemagglutinin neuraminidase genes of simian virus 41 (Genbank Acc. Nos. X64275 or S46730), human parainfluenza virus type 3 (M17641) and Newcastle disease virus (Genbank Acc. No. J03911).

c) Membrane Glycoproteins of the Herpesvirus Family.

Certain members of the Herpesvirdae family are renowned for their potent syncytium-inducing activity. Indeed, Varicella-Zoster Virus has no natural cell-free state in tissue culture and spreads almost exclusively by inducing cell fusion, forming large syncytia which eventually encompass the entire monolayer. gH is a strongly fusogenic glycoprotein which is highly conserved among the herpesvirus; two such proteins are gH of human herpesvirus 1 (Genbank Acc. No. X03896) and simian varicella virus (Genbank Acc. No. U25806). Maturation and membrane expression of gH are dependent on coexpression of the virally encoded chaperone protein gL (Duus et al., Virology, 1995, 210, 429-440). Although gH is not the only fusogenic membrane glycoprotein encoded in the herpesvirus genome (gB has also been shown to induce syncytium formation), it is required for the expression of virus infectivity (Forrester et al., J. Virol., 1992, 66, 341-348). Representative genes encoding gB are found in human (Genbank Acc. No. M14923), bovine (Genbank Acc. No. Z15044) and cercopithecine (Genbank Acc. No. U12388) herpesviruses.

d) Membrane Glycoproteins of Retroviruses.

Retroviruses use a single homo-oligomeric membrane glycoprotein for attachment and fusion triggering. Each subunit in the oligomeric complex is synthesized as a polyprotein precursor which is proteolytically cleaved into membrane-anchored (TM) and extraviral (SU) components which remain associated through covalent or noncovalent interactions. Cleavage activation of the retroviral envelope precursor polypeptide is usually mediated by a Golgi protease during protein transport to the cell surface. There are inhibitory (R) peptides on the cytoplasmic tails of the TM subunits of the envelope glycoproteins of Friend murine leukemia virus (FMLV, EMBL accession number X02794) and Mason Pfizer monkey virus (MPMV; Genbank Acc. No. M12349) which are cleaved by the virally encoded protease after virus budding has occurred. Cleavage of the R peptide is required to activate fully the fusogenic potential of these envelope glycoproteins and mutants lacking the R peptide show greatly enhanced activity in cell fusion assays (Rein et al, J. Virol ., 1994, 68, 1773-1781; Ragheb & Anderson, J. Virol., 1994, 68, 3220-3231; Brody et al, J. Virol. 1994, 68, 4620-4627).

e) MLV Membrane Glycoproteins with Altered Specificity.

Naturally occurring MLV strains can also differ greatly in their propensity for syncytium induction in specific cell types or tissues. One MLV variant shows a strong tendency to induce the formation of endothelial cell syncytia in cerebral blood vessels, leading to intracerebral hemorrhages and neurologic disease. The altered behavior of this MLV variant can be reproduced by introducing a single point mutation in the env gene of a non-neurovirulent strain of Friend MLV, resulting in a tryptophan-to-glycine substitution at amino acid position 120 in the variable region of the SU glycoprotein (Park et al, J. Virol., 1994, 68, 7516-7524).

f) HIV Membrane Glycoproteins.

HIV strains are also known to differ greatly in their ability to induce the formation of T cell syncytia and these differences are known to be determined in large part by variability between the envelope glycoproteins of different strains. Typical examples are provided by Genbank accessions L15085 (V1 and V2 regions) and U29433 (V3 region).

g) Acid-triggered Fusogenic Glycoproteins Having an Altered pH Optimum.

The membrane glycoproteins of viruses that normally trigger fusion at acid pH do not usually promote syncytium formation. However, they can trigger cell-cell fusion under certain circumstances. For example, syncytia have been observed when cells expressing influenza hemagglutinin (Genbank Acc. No. U44483) or the G protein of Vesicular Stomatitis Virus (Genbank Acc. Nos. M21417 and J04326) are exposed to acid (Steinhauer et al, Proc. Natl. Acad. Sci. USA 1996, 93, 12873-12878) or when the fisogenic glycoproteins are expressed at a very high density (Yang et al, Hum. Gene Ther.1995, 6, 1203-1213). In addition, acid-triggered fusogenic viral membrane glycoproteins can be mutated to shift their pH optimum for fusion triggering (Steinhauer et al, Proc. Natl. Acad. Sci. USA 1996, 93, 12873-12878).

h) Membrane Glycoproteins from Poxviruses.

The ability of poxviruses to cause cell fusion at neutral pH correlates strongly with a lack of HA production (Ichihashi & Dales, Virology, 1971, 46, 533-543). Wild type vaccinia virus, an HA-positive orthopoxvirus, does not cause cell fusion at neutral pH, but can be induced to do so by acid pH treatment of infected cells (Gong et al, Virology, 1990, 178, 81-91). In contrast, wild type rabbitpox virus, which lacks a HA gene, causes cell fusion at neutral pH. However, inactivation of the HA or SPI-3 (serpin) genes in HA-positive orthopoxviruses leads to the formation of syncytia by fusion of infected cells at neutral pH (Turner & Moyer, J. Virol. 1992, 66, 2076-2085). Current evidence indicates that the SPI-3 and HA gene products act through a common pathway to control the activity of the orthopoxvirus fusion-triggering viral glycoproteins, thereby preventing fusion of cells infected with wild type virus.

i) Membrane Glycoproteins of Other Replicating Viruses.

Replicating viruses are known to encode fusogenic viral membrane glycoproteins, which viruses include but are not limited to mumps virus (hemagglutinin neuraminidase, SwissProt P33480; glycoproteins F1 and F2, SwissProt P33481), West Nile virus (Genbank Acc. Nos. M12294 and M10103), herpes simplex virus (see above), Russian Far East encephalitis, Newcastle disease virus (see above), Venezuelan equine encephalomyelitis (Genbank Acc. No. L044599), rabies (Genbank Acc. No. U11736 and others), vaccinia (EMBL accession X91135) and varicella (GenPept U25806; Russell, 1994, Eur. J. Cancer, 30A, 1165-1171).

In addition to virally-derived FMGs used in the form normally present in the virus, viral FMGs used in the invention may be engineered or modified to optimize their characteristics for therapeutic use (e.g. enhanced fusogenic activity, or introduction of novel binding specificities to assist in targeting of the fusion hybrid) as disclosed below.

Modifications of FMGs Leading to Enhanced Fusogenicity

Truncation of the cytoplasmic domains of a number of retroviral and herpesvirus glycoproteins has been shown to increase their fusion activity, sometimes with a simultaneous reduction in the efficiency with which they are incorporated into virions (Rein et al, J. Virol. 1994, 68. 1773-1781; Brody et al, J. Virol. 1994, 68, 4620-4627; Mulligan et al, J. Virol. 1992, 66, 3971-3975; Pique et al, J. Virol. 1993, 67, 557-561; Baghian et al, J. Virol. 1993, 67, 2396-2401; Gage et al, J. Virol. 1993, 67, 2191-2201). Further, transmembrane domain swapping experiments between MLV and HTLV-1 have shown that envelopes which are readily fusogenic in cell-to-cell assays and also efficiently incorporated into virions may not necessarily confer virus-to-cell fusogenicity (Denesvre et al., J. Virol. 1996, 70, 4380-4386).

Modifications of FMGs to Obtain Enhanced Selectivity of Syncytium Induction

The selectivity of syncytium induction by a viral FMG may be modified if so desired by fusing targeting moieties to the FMG that provide novel binding specificities. Novel binding specificities may be introduced into the FMG such that the modified FMG may recognize a selected receptor or antigen on a target cell, and thereby target the fusogenic activity to a specific cell type that expresses the targeted receptor or antigen. The altered glycoprotein may be tissue selective, as any tissue may give rise to a malignancy. Possible target antigens are preferentially expressed on breast, prostate, colon, ovary, testis, lung, stomach, pancreas, liver, thyroid, hemopoietic progenitor, T cells, B cells, muscle, nerve, etc. Additional possible target antigens include true tumor-specific antigens and oncofetal antigens. For example, B lymphocytes are known to give rise to at least 20 different types of hematological malignancy, with potential target molecules including CD10, CD19, CD20, CD21, CD22, CD38, CD40, CD52, surface IgM, surface IgD, idiotypic determinants on the surface of Ig, MHC class II, receptors for IL2, IL4, IL5, IL6, etc. Fusogenic membrane glycoproteins may be modified so as to contain receptor binding components of any ligand, for example, including monoclonal antibodies, naturally occurring growth factors such as interleukins, cytolines, chemokines, adhesins, integrins, neuropeptides, and non-natural peptides selected from phage libraries, and peptide toxins such as conotoxins, and agatoxins.

2. Non-viral Fusogenic Membrane Proteins.

Cell-cell fusion occurs between some mammalian cells without the influence of viral membrane glycoproteins. For example, sperm and egg fusion occurs at fertilization. The fusogenic membrane protein carried by sperm has been identified as fertilin b, and the egg cell surface receptor is alpha-6, beta-1 integrin (Chen & Sampson, 1999, Chem. Biol. 6: 1-10).

Other examples of cell fusion occurring in mammalian systems include the fusion of myoblasts in skeletal and cardiac muscle, which function as viable syncytia. Further, in the early stages of pregnancy, blastocyst attachment to the uterus involves the adhesion of the trophoblast to the uterine epithelial surface. Fusion between adjacent epithelial cells precedes the initial attachment of the blastocyst, and is followed by fusion between the trophoblast and the epithelium. A member of the cellular metalloproteinase/disintegrin family, MDC9, has integrin-binding, metalloproteinase and fusogenic functions and has been implicated in epithelial cell fusion that precedes trophoblast fusion. Also during pregnancy, the trophoblast, supporting the main functions of the placenta, develops from the fusion of cytotrophoblastic cells into a syncytiotrophoblast. The fusion of cytotrophoblastic cells is complex, and involves factors and pathways common to regulation of the apoptotic cascade, such as Bc1-2, Mc1-1 and topoisomerase IIa (Huppertz et al., 1998, Histochem Cell. Biol. 110: 495-508), as well as cAMP-dependent protein kinase type IIa (Keryer et al., 1998, J. Cell Sci. 111: 995-1004).

It is comtemplated that the cell fusion-promoting activities of proteins involved in sperm-egg fusion, myoblast fusion and cytotrophoblast syncytial formation can be exploited in the cell fusion methods of the invention.

B. Tumor-specific Promoters Useful According to the Invention.

Tumor-specific promoters are utilized in the nucleic acid constructs and methods of the invention to provide strong expression of fusogenic polypeptides, cytotoxic gene products, cytokines, and in some preferred embodiments, a recombinase, in a substantially tumor-restricted manner. As used herein, the term “strong expression” means that the level of a transcript generated from a given promoter results in a steady-state level of transcript of at least about 100 molecules per cell, 250 molecules per cell, or 500 molecules per cell or more, up to 1,000, 5,000, 10,000 or more molecules per cell. As used herein, the term “tumor-restricted manner” means that the transcription of a gene is at least 5-fold, 10-fold, 25-fold, 50-fold, 100-fold, 500-fold, 1,000-fold or more times more active in an appropriate tumor cell, in terms of the amount of transcription directed by the promoter, than in a non-fetal, non-tumor cell of the same lineage. The selection of a tumor-specific promoter for use in a method of the invention clearly depends upon the nature of the tumor being treated. Put simply, in order to be effective, the selected tumor-specific promoter must be active in the tumor being targeted. It is well within the ability of one skilled in the art to determine the activity of a given tumor-specific promoter in a given tumor. Because antibody preparations specific for tumor-specific antigens are widely available, straightforward immunoassays known in the art are applicable to evaluating the activity of tumor-specific promoters in a given tumor. Further, methods described below for assessing the activity of tissue-specific promoters may be readliy adapted to assess the activity of tumor specific promoters in a given tumor or tumor type.

In addition to being active in tumor cells, a tumor-specific promoter useful in the constructs and methods of the invention should be substantially inactive in non-transformed, non-malignant or non-tumor cells. There are a number of known tumor-specific promoters which are suitable for incorporation into the nucleic acid constructs and methods of the invention. The expression of a number of antigens is associated with specific types of tumors. Each of these so-called “tumor antigens” is driven by a promoter that is active in one or more types of tumor but substantially inactive in non-tumor cells. The promoters for tumor antigens are therefore good candidates for tumor-specific promoters according to the invention. It is noted that the tumor-specific promoters useful in the invention are preferably those from human genes, but that a tumor-specific promoter from any species (e.g., bovine or murine promoters) is acceptable according to the invention as long as it drives the transcription of operably linked sequences in a tumor specific manner in the species being treated.

Tumor antigens include, but are not limited to, prostate specific antigen (PSA; Osterling, 1991, J. Urol., 145: 907-923), epithelial membrane antigen (multiple epithelial carcinomas; Pinkus et al., 1986, Am. J. Clin. Pathol. 85: 269-277), CYFRA 21-1 (lung cancer; Lai et al., 1999, Jpn. J. Clin. Oncol. 29: 421-421) and Ep-CAM (pan-carcinoma; Chaubal et al., 1999, Anticancer Res. 19: 2237-2242).

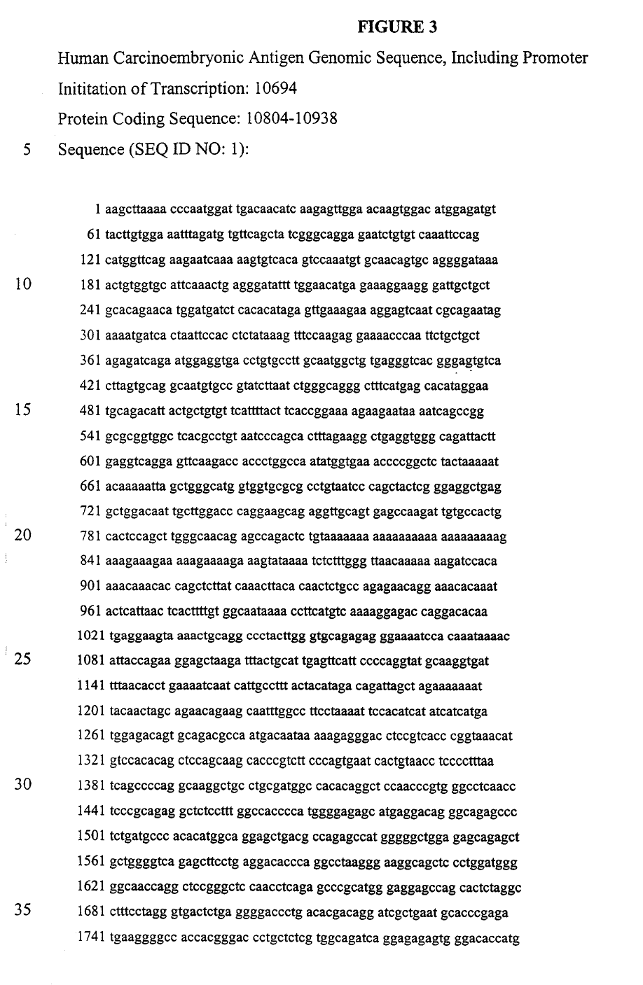

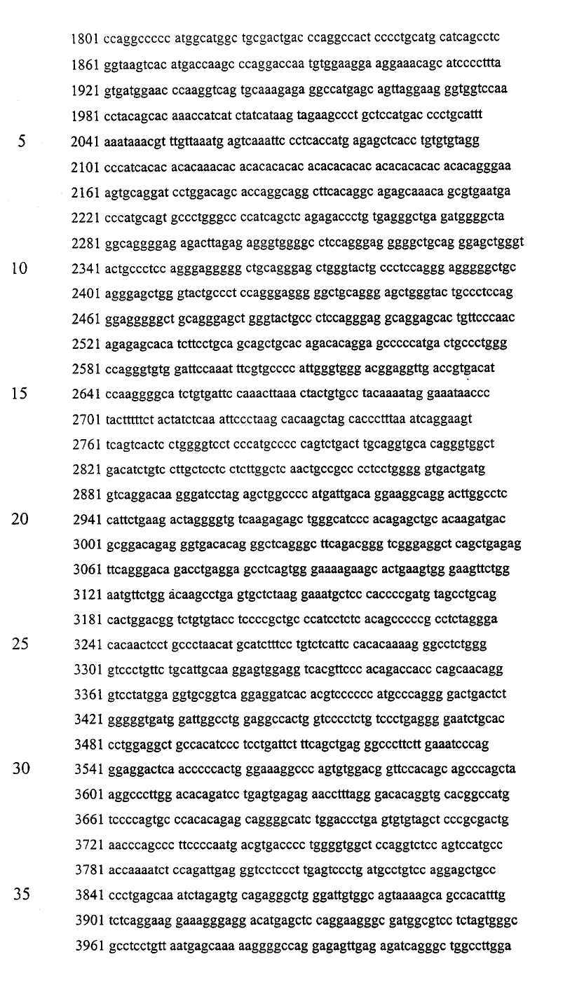

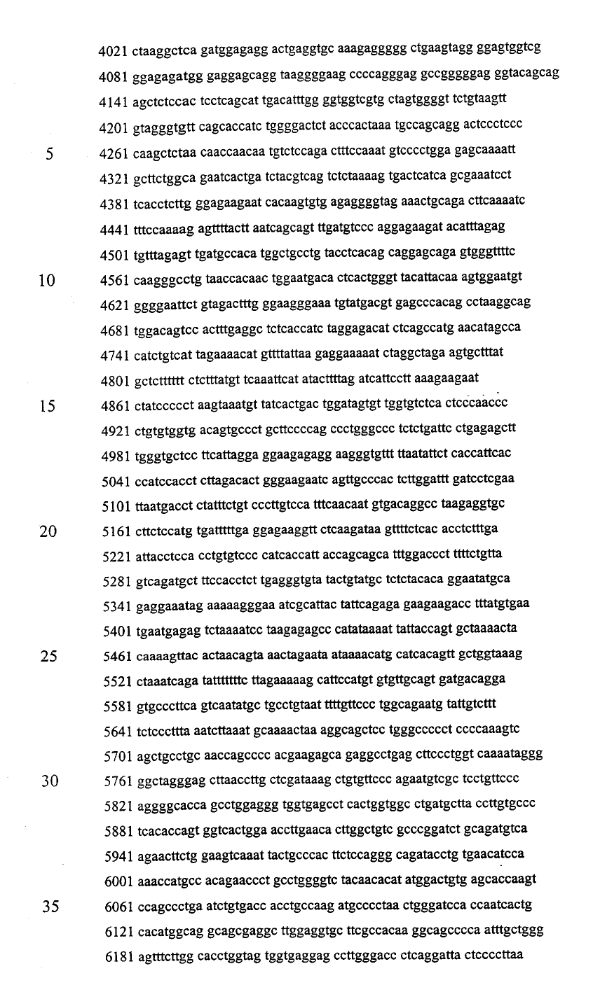

Also included in the category of tumor-specific promoters are a number of promoters for gene products or antigens that are expressed in normal, non-transformed tissues but are not normally expressed in fully differentiated tissues or in tissues of the adult organism. These so-called “oncofetal antigens” include polypeptides normally expressed only during development that are re-expressed in tumor tissues. Non-limiting examples include the liver-specific protein alphafetoprotein (AFP), which is normally expressed in embryonic tissues of the yolk sac, liver, and gastrointestinal tract but is also frequently expressed in tumors of the liver and male germ cells. The AFP promoter/enhancer is therefore an example of a suitable tumor-specific promoter or control element for use in the constructs and methods of the invention. The 5′ flanking sequence of the alpha-fetoprotein gene contains transcription control units with characteristics of enhancers. The enhancer activity is cell-specific in that it occurs in hepatoma cells producing AFP, but not in non-AFP-producing hepatoma or non-hepatoma cells. The active elements can direct reporter expression in conjunction with the SV40 promoter in an orientation-and position-independent manner. The enhancer activity resides in the 400 base pair region between 3.3 and 2.9 kb upstream of the AFP gene. This region and proximal upstream regions contain multiple enhancer ‘core’-like sequences. (GenBank Accession Nos: L34019, human promoter region; J02693, human promoter and enhancer; see also Watanabe et al., 1987, J. Biol. Chem. 262: 4812-4818; Saiki et al., 1985, J. Biol. Chem. 260: 5055-5060; Sawadaishi et al., 1988, Mol. Cell. Biol. 8: 5179-5187; Nakabayashi et al., 1989, J. Biol. Chem. 264: 266-271; Nakabayashi et al., 1991, Mol. Cell. Biol. 11: 5885-5893; and Saegusa et al., 1994, Tumor Marker Oncol. 9: 29-34). Another non-limiting example is carcinoembryonic antigen (CEA), which is normally expressed in embryonic tissues of the gut, pancreas and liver, but which is frequently expressed in carcinomas of the colon, pancreas, lung, stomach and breast. The CEA promoter is therefore an example of a suitable tumor-specific promoter for use in the constructs and methods of the invention (GenBank Accession No: Z21818; see also Richards et al., 1993, DNA Seq. 4: 185-196; Schrewe et al., 1990, Mol. Cell. Biol. 10: 2738-2748; and Richards et al., 1995, Hum. Gene Ther. 6: 881-893). The sequence of the human CEA promoter and coding sequences is provided in FIG. 3.

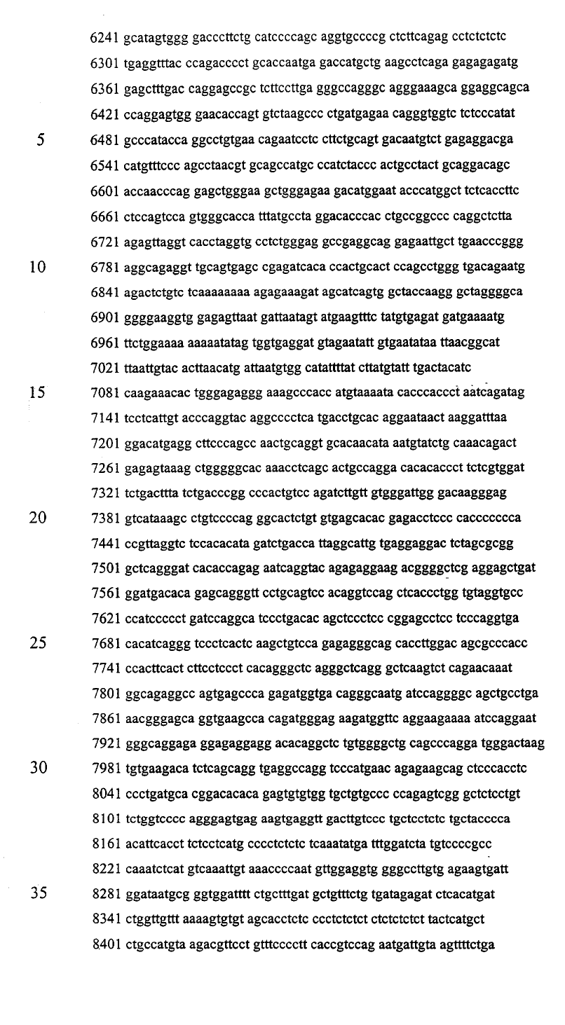

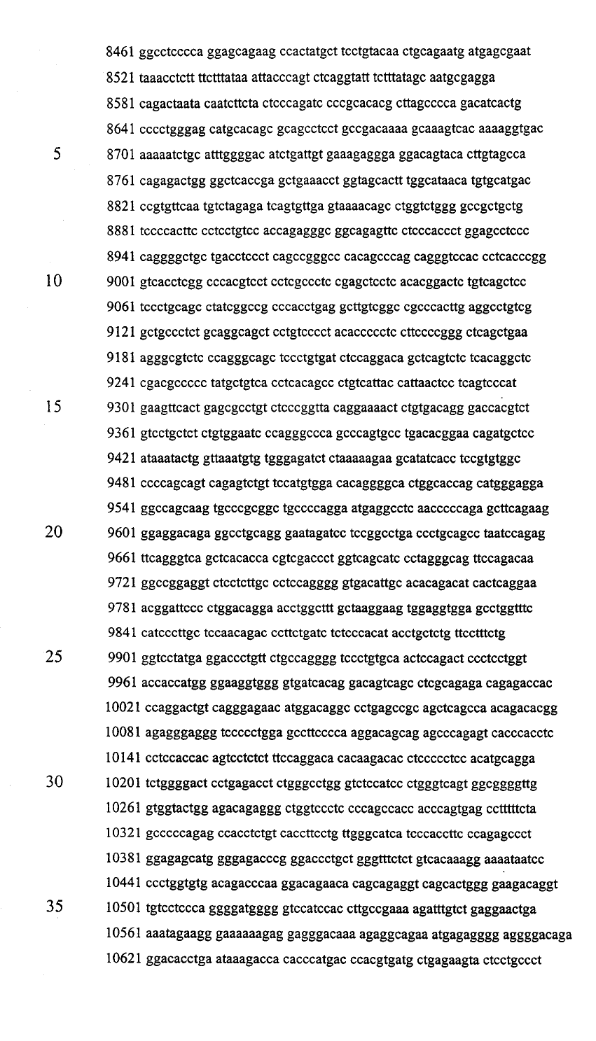

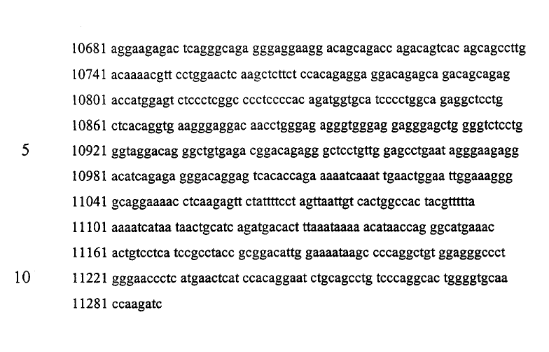

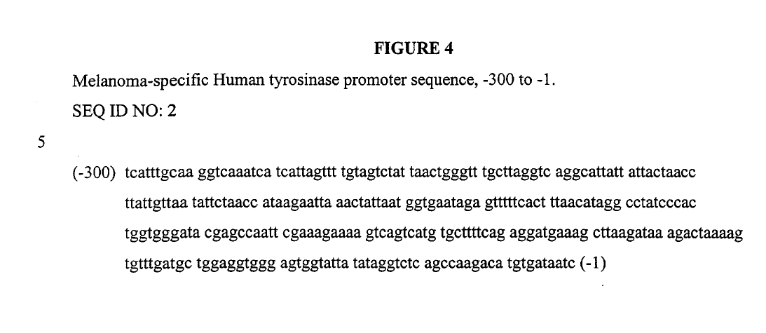

Another tumor-specific promoter that has been described includes the promoter for human tyrosinase, referred to herein as “Tyr300,” which has exceptional specificity for melanoma cells and corresponds to bases −300 to −1 of the tyrosinase gene (SEQ ID NO: 2, shown in FIG. 4; Bentley et al. (1994) Mol. Cell. Biol. 14: 7996-8006). Others include the alpha fetoprotein (hepatocellular carcinomas; Ghebranious et al. (1995) Mol. Reprod. Dev. 42: 1-6), erb-B2 (breast cancer; Pandha et al. (1999) J. Clin. Oncol. 17: 2180), and myelin basic protein (glioma cells; see Shinoura et al. (1999) Cancer Res. 59: 5521-5528) promoters.

Other oncofetal tumor antigens include, but are not limited to, placental alkaline phosphatase (GenBank Accession Nos.: X66946 and X66947 (both human); see also Deonarain et al., 1997, Protein Eng. 10: 89-98; Travers & Bodmer, 1984, Int. J. Cancer 33: 633-641), sialyl-Lewis X (adenocarcinoma, Wittig et al., 1996, Int. J. Cancer 67: 80-85), CA-125 and CA-19 (gastrointestinal, hepatic, and gynecological tumors; Pitkanen et al., 1994, Pediatr. Res. 35: 205-208), TAG-72 (colorectal tumors; Gaudagni et al., 1996, Anticancer Res. 16: 2141-2148), epithelial glycoprotein 2 (pan-carcinoma expression; Roovers et al., 1998, Br. J. Cancer. 78: 1407-1416), pancreatic oncofetal antigen (Kithier et al., 1992, Tumor Biol. 13: 343-351), 5T4 (gastric carcinoma; Starzynska et al., 1998, Eur. J. Gastroenterol. Hepatol. 10: 479-484,; alphafetoprotein receptor (multiple tumor types, particularly mammary tumors; Moro et al., 1993, Tumour Biol. 14: 11-130), and M2A (germ cell neoplasia; Marks et al., 1999, Brit. J. Cancer 80: 569-578). All of the references from this and the preceding 3 paragraphs are incorporated herein in their entirety by reference. The promoters/enhancers driving these genes are considered suitable promoters for use in the constructs and methods of the invention, as long as they drive the tumor-specific expression of linked sequences when introduced to tumor cells. The promoters/enhancers for known tumor-specific genes may be isolated, if so desired, by one of skill in the art according to methods similar to those described below for the isolation of tissue-specific promoters.

C. Tissue-specific Promoters Useful According to the Invention.

Tissue-specific promoters are utilized in the nucleic acid constructs and methods of the invention to provide tissue-specific expression of a recombinase in non-tumor tissues surrounding or adjacent to a tumor being targeted with a method of the invention, in a substantially tumor-restricted manner. The selection of a tissue-specific promoter for use in a method of the invention clearly depends upon the nature of the tumor being treated. That is, the tissue-specific promoter used must be active in the non-transformed cells of the tissue that gave rise to the tumor. A large number of tissue-specific promoters are known. For example, there is an extensive list of cis-acting control elements exhibiting tissue-specific regulation provided in the review of regulatable vectors for genetic therapy by Miller & Whelan (1997, Human Gene Ther. 8: 803-815). Further tissue-specific regulatory sequences are described by Miller & Vile (1995, FASEB J. 9: 190-199). Both of these references are incorporated herein in their entirety by reference, and Table 1 lists the promoters they describe and the published references containing those promoter sequences, which are also incorporated herein by reference. As stated above for tumor-specific promoters, the term “tissue-specific promoter”, as used herein, includes all elements necessary to drive the tissue-specific expression of an operably linked gene sequence. Thus, the term includes not only the basal promoter elements, but also those elements such as enhancers and even silencers necessary to confer tissue-specific expression upon the operably linked gene sequence.

TABLE 1

| | TISSUE | REFERENCE FOR |
|---|---|---|
| GENE | SPECIFICITY | PROMOTER SEQUENCE |
| Transferrin | Brain | Bowman et al, 1995 Proc. Natl. |
| | | Acad. Sci. USA 92,12115-12119 |
| Synapsin I | Neurons | Schoch et al, 1996 J. Biol. Chem. |
| | | 271, 3317-3323 |
| Necdin | Post-mitotic | Uetsuki et al., 1996 J. Biol. Chem. |
| | neurons | 271, 918-924 |
| Neurofilament | Neurons | Charron et al., 1995 J. Biol. Chem. |
| light | | 270, 30604-30610 |
| Acetylcholine | Neurons | Wood et al., 1995 J. Biol. Chem. |
| receptor | | 270, 30933-30940 |
| Potassium | High-frequency | Gan et al., 1996 J. Biol. Chem |
| channel | firing neurons | 271, 5859-5865 |
| Chromogranin A | Neuroendocrine | Wu et al., 1995 A. J. Clin. Invest. |
| | cells | 96, 568-578 |
| Von Willebrand | Brain | Aird et al, 1995 Proc. Natl. Acad. |
| factor | endothelium | Sci. USA 92, 4567-4571. |
| flt-1 | Endothelium | Morishita et al, 1995 J. Biol. |
| | | Chem. 270, 27948-27953 |
| Preproendo- | Endothelium, | Harats et al., 1995 J. Clin. Invest. |
| thelin-1 | epithelium, | 95, 1335-1344 |
| | muscle | |
| GLUT4 | Skeletal muscle | Olson and Pessin, 1995 J. Biol. |
| | | Chem. 270, 23491-23495 |
| Slow/fast | Slow/fast twitch | Corin et al., 1995 Proc. Natl. |
| troponins | myofibres | Acad. Sci. USA 92, 6185-6189 |
| a-Actin | Smooth muscle | Shimizu et al., 1995 J. Biol. Chem. |
| | | 270, 7631-7643 |
| Myosin | Smooth muscle | Kallmeier et al., 1995 J. Biol. |
| heavy chain | | Chem. 270, 30949-30957 |
| E-cadherin | Epithelium | Hennig et al., 1996 J. Biol. Chem. |
| | | 271, 595-602 |
| Cytokeratins | Keratinocytes | Alexander et al., 1995 B. Hum. |
| | | Mol. Genet. 4, 993-999 |
| Transglutaminase | Keratinocytes | J. Lee et al, 1996 J. Biol. Chem. |
| 3 | | 271, 4561-4568 |
| Bullous | Basal | Tamai et al., 1995 J. Biol. Chem. |
| pemphigoid | Keratinocytes | 270, 7609-7614 |
| antigen | | |
| Keratin 6 | Proligernting | Ramirez et al, 1995 Proc. Natl. |
| | epidermis | Acad. Sci. USA 92, 4783-4787 |
| Collagen a1 | Hepatic stellate | Houglum et al., 1995 J. Clin. |
| | cell skin/tendon | Invest. 96, 2269-2276 |
| | fibroblasts | |
| Type X | Hypertrophic | Long & Linsenmayer, 1995 Hum. |
| collagen | Chondrocytes | Gene Ther. 6, 419-428 |
| Factor VII | Liver | Greenberg et al, 1995 Proc. Natl. |
| | | Acad. Sci. USA 92, 12347-12351 |
| Fatty acid | Liver, adipose | Soncini et al., 1995 J. Biol. Chem. |
| synthase | tissue | 270, 30339-30343 |
| Carbamoyl | Portal vein | Christoffels et al., 1995 J. Biol. |
| phosphate | hepatocytes | Chem. 270, 24932-24940 |
| synthetase I | Small intestine | |
| Na—K—Cl | Kidney (loop of | Igarashi et al., 1996 J. Biol. Chem. |
| transporter | Henle) | 271, 9666-9674 |
| Scavenger | Macrophages, | Horvai et al., 1995 Proc. Natl. |
| receptor A | foam cells | Acad. Sci. USA 92, 5391-5395 |
| Glycoprotein | Megakaryocytes, | Block & Poncz, 1995 Stem Cells |
| IIb | platelets | 13, 135-145 |
| yc chain | Hematopoietic | Markiewicz et al., 1996 J. Biol. |
| | cells | Chem. 271, 14849-14855 |
| CD11b | Mature myeloid | Dziennis et al., 1995 Blood 85, |
| | cells | 319-329 |

In addition to the promoter sequences provided in the references noted in Table 1, one of skill in the art can isolate the promoter for a tissue-specific gene using standard methods known in the art. It is known that a basal promoter lies 5′ of the coding region of a gene and often, but not always, comprises an A/T-rich element (the TATA box) within about 25 base pairs (bp) of the initiation site of transcription, and a CAAT box element about 75 bp upstream of the initiation site. Enhancer and/or silencer elements essential for tissue- or tumor-specific regulation of a promoter are usually located within about 1-5 kb upstream of (i.e., 5′ of) the transcript initiation site(s), but may lie as far as 10 kilobases (kb) upstream or downstream of the initiation site(s).

In order to isolate the promoter sequence of a known tissue-specific gene, one may take the following steps. 1) If a cDNA is available, one may probe a genomic plasmid or bacteriophage library to identify a genomic clone comprising 5′ untranslated sequences and introns. If a cDNA is not available, one may probe a cDNA expression library (e.g., a lambda phage expression library) with an antibody specific for the gene of interest in order to first identify a cDNA that encodes the gene of interest. 2) The clone is then sequenced to identify the coding region, and reporter constructs comprising varying amounts of the sequences 5′ of the coding region linked to a reporter gene are made and tested by transient transfection into appropriate cells in culture (i.e., cells known to express the gene of interest and cells known not to to express the gene of interest). It is again noted that sequences within the coding region or within introns may also be important for tissue-specific regulation, and therefore should also be evaluated in reporter constructs. Deletion analyses performed on a construct found to exhibit the desired specificity of expression allow the identification of those sequences required for tissue-specific expression driven by that promoter. It is also noted that the same procedures apply to the identification of a tumor-specific promoter, with the exception that expression is tested in tumor versus non-tumor cells, preferably, but not necessarily of the same lineage. Methods of carrying out the steps described above for isolating a tissue-specific (or tumor-specific promoter) are well known in the art, and are described, for example, by Sambrook et al. (Molecular Cloning: A Laboratory Manual, Second Edition, 1989, (Cold Spring Harbor Press, Cold Spring Harbor, N.Y.)), and by Ausubel et al. (Current Protocols in Molecular Biology, 1988, John Wiley & Sons, Inc.).

In order to be useful according to the invention, the selected tissue-specific promoter should be substantially inactive in the tumor cells being targeted for killing. The down regulation of tissue specific genes is frequently observed in tumors. For example, hepatic tumors (e.g., hepatocellular carcinomas) frequently show not only activation of the (oncofetal) AFP promoter, but also down-regulation of the fully differentiated-(or adult-) cell-specific albumin promoter. The substantial lack of activity of the selected tissue-specific promoter in cells of the targeted tumor prevents the excision of the FMP cassette from the construct in tumor cells, allowing continued expression of fusogenic activities in those cells.

In order to determine whether a given tissue-specific promoter or control element is appropriate for use in the constructs and methods of the invention, there are at least two different approaches, one using an immunoassay to monitor the protein product of the candidate tissue-specific gene, and the other using a direct assay of the activity of the candidate tissue-specific promoter in tumor cells.

Immunological Approaches to Evaluating a Tissue-specific Promoter According to the Invention:

The first approach involves an immunoassay of tumor cells to determine the presence of the product of the tissue specific gene whose promoter is being evaluated. This may take the form of immunohistochemistry, wherein cells from a tumor biopsy are stained with antibodies specific for the tissue-specific protein. Another format is (Western) immunoblotting or other immunoassay, such as an ELISA or immunoprecipitation assays. Each of these assay formats is well known to those of skill in the art. Specific details are found in the following references: Voller, 1978, Diagnostic Horizons, 2: 1-7, Microbiological Associates Quarterly Publication, Walkersville, Md.; Voller et al., 1978, J. Clin. Pathol., 31: 507-520; U.S. Reissue Pat. No. 31,006; UK Patent 2,019,408; Butler, 1981, Methods Enzymol., 73: 482-523; Maggio, E. (ed.), 1980, Enzyme Immunoassay, CRC Press, Boca Raton, Fla.; Weintraub, B., Principles of radioimmunoassays, Seventh Training Course on Radioligand Assay Techniques, The Endocrine Society, March 1986, pp. 1-5, 46-49 and 68-78).

When using the immunoassay approach, one looks for a substantial lack of staining or other signal (e.g., radiolabel) specific for the product of the tissue specific gene. This means that the staining or signal is at or below the level of background staining observed in morphologically transformed cells when a non-related antibody or pre-immune antibody preparation is used to stain cells from the same tumor. Also, the immunoassay approach should be thought of as a “pre-screening” approach, since it is possible for a tissue-specific gene product to be down-regulated in tumor cells through a post-transcriptional mechanism. That is, it is possible to have down-regulation of the protein product without full down-regulation of transcription. Therefore, while a potentially large number of tissue-specific promoters may be screened by assaying for down-regulation of their protein products in a given tumor using appropriate panels of antibodies, those candidates showing a lack of expression should then be screened according to the gene transcription approach described below, or its equivalent.

2. Reporter Assays for the Evaluation of Tissue Specific Promoters Useful According to the Invention

The activity of a candidate promoter is assessed by linking the candidate promoter sequences to a reporter gene and transfecting the construct into tumor cells. A substantial lack of expression of the reporter is indicative of a tissue-specific promoter that is substantially inactive in the tumor cell. In this case, a substantial lack means that the expression of the reporter is at or below the level of expression from a promoterless reporter construct. Any reporter known in the art may be used, including but not limited to green fluorescent protein (GFP; Ogawa et al., 1995, Proc. Natl. Acad. Sci. U.S. A. 92: 11899-11903), bacterial chloramphenicol acetyltransferase (CAT; Gorman et al., 1982, Mol. Cell. Biol. 2: 1044-1051), luciferase (Luc; Nordeen, 1988, BioTechniques 6: 454-457), and -galactosidase (-gal; Mittal et al., 1995, Virology 210: 226-230).

Alternatively, the candidate promoter may be evaluated by linking it to a recombinase gene sequence on a vector also encoding a constitutively active reporter gene sequence (e.g., luciferase or -galactosidase linked to a cytomegalovirus promoter or other strong, constitutive promoter) that is flanked by sites recognized by the recombinase. This construct is then transfected into tumor cells in parallel with a similar construct lacking functional recombinase sequences. Transfected tumor cells are then monitored for reporter expression. In this assay, reporter expression that is equal in cells transfected with either construct is indicative of a tissue-specific promoter that is not active in the tumor cells. In other words, if the tissue-specific promoter linked to the recombinase is active, reporter expression will decrease relative to reporter expression in cells transfected with a similar construct lacking functional recombinase sequences.

In order to avoid differences in transfection efficiency due to differing sizes of the constructs, it is recommended, although not absolutely necessary, to introduce inactivating mutations to the recombinase gene in the control, non-functional recombinase construct (e.g., introduction of stop codons in one or more reading frames) rather than wholesale deletion of the recombinase coding sequences. One skilled in the art also knows that differences in transfection efficiency may be further controlled through co-transfection of each construct with an equal amount of a separate construct constitutively encoding another reporter molecule.

Tumor cells are preferably primary tumor cells taken from the tumor to be treated, or they may be an established tumor cell line with characteristics (such as a tumor antigen expression profile) similar to the tumor of interest. Methods of primary culture of tumor cells are well known in the art. Transfection of the tumor cells is accomplished by any suitable method known in the art, including, for example, lipid-mediated transfection (“lipofection”), electroporation or calcium phosphate precipitation. Lipofection reagents and methods suitable for transient transfection of a wide variety of transformed and non-transformed or primary cells are widely available. For example,LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

D. Cytokines Useful According to the Invention.

In one aspect of the invention, a gene sequence encoding a cytokine is included on a vector construct of the invention. In order to be useful according to the methods of the invention, the selected cytokine should be active in enhancing or potentiating the cell mediated immune response against a targeted tumor. Proteins able to potentiate the killing of tumor cells include those cytokines or other immunostimulatory proteins that stimulate a cell-mediated anti-tumor immune response by recruiting immune cells to the site of cytokine production.

Cytokines or immunostimulatory proteins useful according to this aspect of the invention include, but are not limited to, the following (the number following each cytokine is the GenBank Accession No. for the sequence encoding the cytokine): IL-1, M28983; IL-2, S77834; IL-3, M14743; IL-4, M13982; IL-5, J03478; IL-6, M54894; IL-7, J04156; IL-12, AF101062; IFN-γ, U10360; and TNF-α, M16441.

The expression of a cytokine or immunomodulatory protein by a construct according to the invention may be assessed in infected cell cultures by means known in the art for assaying the presence of the particular protein. For example, expression of immunomodulatory protein may be evaluated by Western (immunoblot) analysis using antibodies recognizing the specific protein. Other immunoassays, such as ELISAs may be used, or, alternatively, cell-based assays for the activity of the protein may be used as known in the art.

E. Cytotoxic Gene Products Useful According to the Invention.

In one aspect of the invention, a sequence encoding a cytotoxic gene product is included on a vector construct according to the invention. As used herein, the term “cytotoxic gene product refers to a polypeptide that when expressed in a cell results in the death of that cell.

One important class of cytotoxic gene products is the group of enzymes that catalyze the conversion of a non-cytotoxic pro-drug to a cytotoxic agent in cells expressing the enzyme (reviewed by Kwon. 1999, Arch. Pharm. Res. 22: 533-541, incorporated herein by reference). Cytotoxic gene products useful according to the invention include, but are not limited to the following: genes for fusogenic membrane glycoproteins (e.g., VSV-G glycoprotein), Herpes Simplex virus thymidine kinase (HSV TK, which renders cells susceptible to gancyclovir killing; GenBank Accession Nos. U25806, AF135370), cytosine deaminase, which renders cells susceptible to 5-fluorocytosine killing (Westphal et al., 2000, Cancer Gene Res. 7: 97-106, incorporated herein by reference; GenBank Accession No. S56903) and nitroreductase, which renders cells susceptible to CB 1954 killing (Westphal et al., supra; U.S. Pat. No. 5,780,585; and Genbank Accession No. AR018125).

F. Recombination Systems Useful According to the Invention.

The methods of the invention require the use of a site-specific recombinase system. In general, a site-specific recombinase system consists of three elements: two pairs of DNA sequence (the site-specific recombination sequences) and a specific enzyme (the site-specific recombinase). The site-specific recombinase will catalyze a recombination reaction only between two site-specific recombination sequences. That is, excision or recombination requires that the sequence to be excised or recombined be flanked on either side by a sequence recognized and cleavable by a recombinase.

A number of different site specific recombinase systems can be used, including but not limited to the Cre/lox system of bacteriophage P1, the FLP/FRT system of yeast, the Gin recombinase of phage Mu, the Pin recombinase of E. coli, the R/RS system of the pSR1 plasmid, and the Integrase/att system from bacteriophage lambda.

Perhaps the best studied of these are the Integrase/att system from bacteriophage lambda (Landy, A. Current Opinions in Genetics and Devel. 3:699-707 (1993); Landy, A., 1989, Ann. Rev. Biochem. 58: 913), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2u circle plasmid (Broach et al. Cell 29:227-234 (1982)).

The R-RS system from Zygosaccharomyces rouxii (Maeser and Kahmann, 1991, Mol. Gen. Genetics 230: 170-176), like the Cre-loxP and FLP-FRT systems, requires only the protein and its recognition site. The gin-gix recombinase system from bacteriophage Mu selectively mediates DNA inversion between two inversely oriented recombination sites (gix) and requires the assistance of three additional factors: negative supercoiling, an enhancer sequence and its binding protein Fis (Onouchi et al., 1995, Mol. Cell. Biol. 247: 653-660).

The Cre system utilizes the Cre recombinase, which is a 38 lI<a protein, and two 34 bp recombinase target sites, termed loxP (5′-ATAACTTCGTATAGCATACATTATACGAAGT TAT-3′). Recombination can occur between directly repeated loxP sites on the same molecule to excise the intervening DNA segment. See Sauer et al., Proc. Natl. Acad. Sci. USA 85:5166 (1988); Sauer et al., Nuc. Acids Res. 17:147 (1989); Lakso et al., Proc. Natl. Acad. Sci. USA 89:6232; Hoess et al., J. Mol. Biol. 181:351-362 (1985); Abremski et al., Cell 32:1301 (1983); Stemberg et al., J. Mol. Biol. 150:467-486 (1981); and Orban et al., Proc. Natl. Acad. Sci. USA 89:6861 (1992). These references are incorporated herein in their entirety by reference.

The FLP system utilizes the FLP protein and two FLP recombination target sites (termed FRT in the art) that consist of two 13 base pair (bp) inverted repeats and an 8 bp spacer (5′-GAAGTTCCTATACTTTCTAGAGAATAGGAACTTC-3′) (See for example O'Gorman, Science 251:1351 (1991); Jayaram, PNAS USA 82:5875-5879 (1985); Senecof et al., PNAS USA 82:7270 (1985); and Gronostajski et al., J. Biol. Chem. 260:12320 (1985)). All of these references are expressly incorporated in their entirety by reference. It is noted that the FLP/FRT system of yeast has an advantage over the other site specific recombinase systems since it normally functions in a eukaryotic organism (yeast), and is well characterized. The eukaryotic origin of the FLP/FRT system may allow the FLP/FRT system to function more efficiently in eukaryotic cells than the prokaryotic site specific recombinase systems.

Lambda phage Int recombinase site core region DNA sequences include an attR and an attL core sequence. Any two R and L sequences together are required for excisive recombination.

GTTCAGCTTTCKTRTACNAACTSGB (m-attR);
AGCCWGCTTTCKTRTACNAAGTSGB (m-attL);
GTTCAGCTTTGTACAAACTTGT (attR1);
GTTCAGCTTRCTTGTACAAACTTGT (attnR2);
GTTCAGCTTTCTTGTACAAAGTTGG (attR3);
AGCCTGCTTTTTTGTACAAAGTTGG (attL1);
AGCCTGCTTTCTTGTACAAAGTTGG (attL2);
ACCCAGCTTTCTTGTACAAAGTTGG (attL3);
or a corresponding or complementary DNA or RNA
sequence; wherein R = A or G; K = G or T/U;
Y = C or T/U; W = A or T/U; N = A or C or G or T/U;
S = Cor G; and B = C or G or T/U.

F. Vectors Useful According to the Invention.

Any of a number of different types of vector are suitable for use in the methods of the invention. For example, plasmid vectors and viral vectors, including but not limited to retroviral vectors, are useful for carrying and delivering the genetic information necessary for the methods of the invention.

Plasmid Vectors

Plasmids may be used to carry sequences encoding the expression cassettes required for the methods of the invention. A large number of plasmids are known to those skilled in the art. The basic requirements of a plasmid vector useful according to the invention are as follows. Useful mammalian plasmid expression vectors will comprise an origin of replication, a suitable promoter and optional enhancer, and also any necessary ribosome binding sites, a polyadenylation site, splice donor and acceptor sites, transcriptional terrnination sequences, and 5′ flanking nontranscribed sequences. In addition, the expression vectors preferably contain a gene to provide a phenotypic trait for selection of transformed host cells such as dihydrofolate reductase or neomycin resistance for eukaryotic cell culture, or such as tetracycline or ampicillin resistance in E. coli.

Viral Vectors

Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals. Adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). An AAV vector such as that described in Tratschin et al. (1985) Mol. Cell. Biol. 5:3251-3260 can be used to introduce nucleic acid into cells. A variety of nucleic acids have been introduced into different cell types using AAV vectors (see for example Hermonat et al. (1984) Proc. Natl. Acad. Sci. USA 81:6466-6470; and Tratschin et al. (1985) Mol. Cell. Biol. 4:2072-2081).

When expressed concurrently in the same cell, measles virus F and H glycoproteins can mediate cell-cell fusion with neighboring cells, provided the neighboring cells express the measles virus receptor (CD46). Human cells express the CD46 measles virus receptor, whereas murine cells do not. Retroviral vectors expressing the measles virus F and H proteins, and carrying constructs encoding a recombinase are useful in the methods of the invention. The vectors are used to direct expression of the fusogenic membrane protein (FMP) in a cell as described herein. The construction of a retroviral vector comprising measles virus F and H proteins and the subsequent production of infectious viral particles is described below.

1. Construction of retroviral vector plasmid coding for measles virus F and H glycoproteins is described in detail in WO98/40492, hereby incorporated by reference. Briefly, a plasmid is constructed using standard cloning methods. The plasmid, from left to right (representing 5′ to 3′ on a genetic map, contains an LTR (Moloney murine leukaemia virus long terminal repeat), a Moloney murine leukaemia virus packaging signal, an IRES (poliovirus internal ribosome entry site), a measles virus H glycoprotein coding sequence, a measles virus F glycoprotein coding sequence, and a phleomycin resistance marker. The vector backbone is either pUC or pBR322-based. The coding sequence of the measles virus H gene is cloned from pCGH5 (Cathomen et al, 1995, Virology, 214, 628-632), into the NotI site of the retroviral vector plasmid pGCP (which contains the poliovirus internal ribosome entry site flanked by NotI and ClaI cloning sites). The measles virus F gene is then cloned from pCGF (Cathomen et al, 1995, Virology, 214, 628-632) into the ClaI site of the same vector, 5′ of the internal ribosome entry site to produce the vector named pHF. A phleomycin selectable marker gene is then introduced into the vector 5′ of the 5′ LTR.

2. Preparation of retroviral vector stocks.

The plasmid pHF is transfected into amphotropic retroviral packaging cell lines which were derived from murine fibroblasts. Suitable packaging cell lines are widely available and include the NIH3T3-derived cell lines PA317 and GP+env AM12. Stably transfected packaging cells are selected in phleomycin 50 ug/ml and used as a source of HF retroviral vectors capable of efficiently transferring the measles virus F and H genes to human and murine target cells.

G. Administering a Nucleic Acid Vector According to the Invention.

As used herein, the term “administering” refers to the introduction of recombinant nucelic acids, viruses, or cells of the invention to an individual for therapeutic purposes. Recombinant nucleic acids, viruses or cells may be administered, for example, intravenously, intraperitoneally, or even directly into a tumor.

As used herein, the term “physiologically acceptable carrier” refers to a solution or composition in which nucleic acid vectors, viruses or cells of the invention may be suspended to allow administration (e.g., intravenously, intraperitoneally, etc.) of the vectors, viruses or cells to an individual. A physiologically acceptable carrier or diluent will generally be isotonic and will often be buffered; a large number of acceptable diluents or carriers are known in the art. As non-limiting examples, saline and phosphate-buffered saline are acceptable diluents or carriers. It is specifically noted that tissue culture medium containing added bovine or equine serum is not a physiologically acceptable diluent or carrier according to the invention.

A nucleic acid vector may be administered as naked plasmid DNA that is injected directly into a tumor. The efficiency of uptake of a nucleic acid vector can be enhanced by, for example, packaging the DNA in liposomes or with another targeting agent and then directly injecting the complex into the tumor.

Viral vectors may be introduced to a tumor by infection with recombinant viruses. Viral vectors of use in the invention may be targeted to a specific tissue or tumor type using methods as described herein or as known in the art. Alternatively, because the present invention includes a mechanism to prevent the expression of characteristics that might be harmful to tissues other than the targeted tumor, it is not as essential that viral vectors be radically limited in their tissue tropism or infection spectrum. Therefore, viral vectors of the invention may be administered either systemically (i.e., intravenously) or locally. For local administration, injection directly into the tumor is preferred.

Viral vectors may also be introduced to a tumor by transfection of tumor cells with a recombinant viral vector. In this instance, cells from the tumor being targeted (i.e., autologous tumor cells) are placed in culture and then transfected with the recombinant viral vector(s) before being reintroduced to the tumor by injection. Methods of transfecting cultured tumor cells include lipofection, electroporation, and calcium phosphate precipitation, among others. Lipofection is particularly applicable due to its high efficiency and relatively low toxicity, and may be performed according to methods well known in the art using, for example, kits and reagents described elsewhere herein. Following transfection of autologous tumor cells with one or more recombinant viral vectors of the invention, transfected cells may be either directly administered to the patient by intratumor injection, or those cells expressing viral markers (including, for example, a selectable marker such as GFP or an antibiotic resisitance gene) may be selected using the appropriate method (e.g., FACS or antibiotic treatment) in order to enrich for cells that actually received and express the construct(s). Enriched or selected populations of transfected tumor cells are then administered in the same manner as non-selected populations.

For direct infection of tumor cells in vivo, dosages of recombinant viruses necessary to observe an effect will vary with the exact vector employed and the type of tumor being targeted. Generally, however, dosages effective to halt or slow the growth of a tumor, reduce the size of a tumor or to reduce the number of malignant cells will range from 1×10⁶ infective particles to 1×10¹⁰ infective particles. In some instances it may be advantageous to administer more than one dose of recombinant virus. For example, virus may be administered two, three or more times, and the timing of the repeat doses may be on the order of several hours to 1, 2, or 3 days or more, up to and including a week, a month, or more, depending on the response observed. Decisions regarding dosages and the frequency of any repeat dosages are best made by the administering physician based upon the individual tumor(s) being treated and the initial results of the treatment. Clinical parameters monitored to evaluate the progress or success of the treatment are discussed below in the section “Assessing the Anti-tumor Effect of Treatment Methods According to the Invention”.

For the administration of autologous tumor cells transfected with a recombinant viral vector of the invention, about 10⁶ to about 10⁸ transfected cells are administered by direct intratumor injection. Decisions regarding dosages and repeat dose size and/or frequency will be dependent upon the individual case being treated and the response to the initial treatment with modified autologous tumor cells carrying a vector according to the invention.

H. Amplification of Anti-tumor Therapy According to the Invention.

One aspect of the invention relates to a method of reducing the size of a tumor that is also well suited for use in conjunction with other anti-tumor treatment approaches. That is, the method is useful not only as a single method of killing tumor cells, but can be used to amplify the killing of tumor cells by a separate anti-tumor approach. In this aspect of the invention, macrophages are transfected with an expression cassette system comprising at least three expression cassettes: 1) a nucleic acid sequence encoding a syncytium-inducing polypeptide, operably linked to an HRE, wherein the whole cassette or at least the syncytium-inducing polypeptide coding sequences are flanked by sites recognized by a recombinase; 2) a nucleic acid sequence encoding a cytokine or a cytotoxic gene product operably linked to a tumor-specific promoter; and 3) a nucleic acid sequence encoding the recombinase that recognizes the sites flanking the first cassette or its coding sequence, such sequence operably linked to a tumor-specific promoter. The tumor-specific promoter linked to the recombinase may be the same as or different from the tumor-specific promoter linked to the cytokine or cytotoxic gene product coding sequence. It is essential, however, that the tumor-specific promoter or promoters selected be active in the tumor cell type being targeted.

Appropriate cytokines, cytotoxic gene products and recombinases may be selected by those of skill in the art, and are discussed herein above.

In this aspect of the invention, macrophages transduced with one or more nucleic acid constructs comprising at least these three expression cassettes are administered to a patient. The macrophages are preferably originally obtained from the patient being treated (i.e., autologous macrophages), but may also be obtained from other individuals. Macrophages may be isolated from peripheral blood, or, alternatively, from alveolar lavage fluid or from peritoneal lavage fluid, according to methods known in the art. Methods of introducing nucleic acid constructs to macrophages are known in the art, and include, for example, transfection (e.g., by liposome mediated DNA transfer or lipofection, electroporation, calcium phosphate precipitation, etc.) and infection with recombinant viral vectors. The chosen vector(s) may optionally carry a selectable marker, such as antibiotic resistance or a cassettte driving expression of a fluorescent polypeptide, allowing identification of successfully transfected cells.

Following introduction of the nucleic acid expression cassettes into the macrophages and any desired selection (e.g., by antibiotic resistance or fluorescence activated cell sorting) for those that received the cassettes, the genetically modified macrophages are administered to a patient. The preparation of the therapeutic composition comprises the steps of preparing the modified macrophages and placing them in admixture with a physiologically acceptable diluent. The concentration of modified macrophages in the preparation will vary, depending upon the chosen route of administration. For example, local (e.g., intratumor) administration requires higher concentrations of macrophages than systemic (e.g., intravenous) administration because the optimal volume of a preparation injected into a tumor is generally smaller than the optimal volume for intravenous delivery. For intratumor delivery, modified macrophages of the invention are suspended in an acceptable diluent at about 1×10⁶ to 1×10⁸ cells per ml, and 0.2 to 5 ml of modified macrophage suspension are administered. For systemic delivery, modified macrophages are suspended in an acceptable diluent at about 1×10³ to 1×10⁷ cells per ml, and 10 ml to 1 liter of cell suspension is administered.

According to this aspect of the invention, modified macrophages of the invention may be administered once, or a number of times, for example, two, three, five, ten or more times. The frequency of any repeat dosages may be determined by the practitioner on the basis of the response to the therapy. Modified macrophages of the invention may be administered as a primary form of tumor treatment, or they may be administered in conjunction with another anti-tumor treatment. When administered in conjunction with another antitumor treatment method, the modified macrophages of the invention may be administered either concurrently with the other selected treatment method or consecutively.

Although not meant to be limited to such a use, the described method involving administration of genetically modified macrophages is particularly well suited to amplifying the killing of tumor cells induced by methods that involve formation of syncytia. The natural affinity of macrophages for syncytia enhances the killing of tumor cells by methods that induce tumor cell syncytia formation. It is also likely, however, that immune cells, particularly macrophages, are present or recruited in relatively large concentrations in the vicinity of a tumor that is undergoing cell killing, regardless of the killing mechanism. As such, the administration of genetically modified macrophages of the invention also leads to enhanced killing of those tumor cells.

I. Assessing the Anti-tumor Effect of Treatment Methods According to the Invention.

The efficacy of treatment of a tumor with any of the methods of the invention may be evaluated by monitoring the size of a tumor (in the case of solid tumors) or the number of tumor cells in a sample of a given size (tumor cell load, for non-solid tumors). Tumor size or tumor cell load may be monitored according to any of a number of means known in the art, including external palpation, ultrasound, magnetic resonance imaging, or through tumor imaging techniques specific to a given tumor type, such as illumination with a labeled tumor-antigen-specific antibody. Tumor growth is considered to be halted or arrested according to the invention if the size of a tumor or the number of tumor cells in a sample of a given size does not increase over time. A tumor is considered to be reduced in size or tumor cell load if it is at least 10%, 20%, 30%, 50%, 75%, 90% smaller (or less abundant) or more, including 100% smaller (that is, the complete absence of tumor cells) than it was immediately prior to the commencement of treatment.

The efficacy of treatment involving the combination of one antitumor approach with a method involving the administration of genetically modified macrophages according to the invention may be monitored in several ways. First, the rate of shrinkage of the tumor or tumor cell load may be monitored before and after administration of the modified macrophages. An increase in the shrinkage rate by 10%, 20%, 50% or more is indicative of effective enhancement of tumor cell killing. Second, tumor biopsies taken after the administration of macrophages may be compared with biopsies taken before commencement of the modified macrophage treatment. Biopsies are examined for evidence of increased immune cell activity, for example, increased cytokine concentrations as determined by immunoassay, or an increased number of infiltrating immune cells as evidenced by standard methods of immunohistochemistry. An increase of 10%, 20%, 50% or more in cytokine concentrations or the number of infiltrating immune cells in a tumor tissue biopsy is indicative of effective treatment using genetically modified macrophages according to the invention.

### EXAMPLES

### Example 1

### Continued Expression of a Fusogenic Membrane Protein Gene is Required for Ongoing Cell Fusion

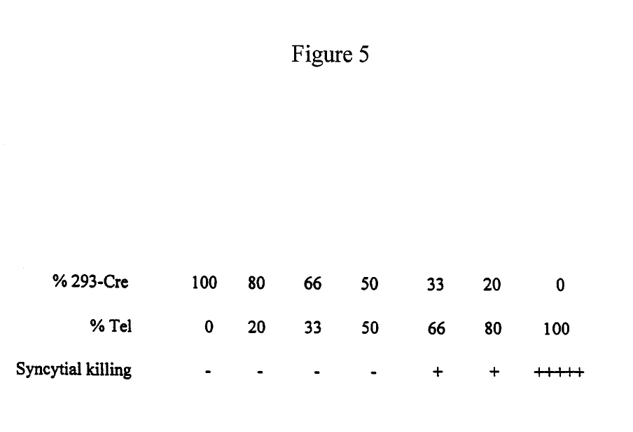

In order to test whether down-regulation of FMP expression by Cre-mediated excision would halt syncytia formation and the ensuing cell death, the following experiment was performed. A CMV-loxP-GALV-loxP vector was transfected into a 1:1 mixture of the Te1.CeB6 (LacZ+) and 293 cell lines. Under these circumstances, extensive cytotoxicity of both cell lines was observed. However, when transfected Te1.CeB6 cells were mixed at different proportions with 293 cells stably expressing the Cre recombinase (293Cre), fusion was arrested, or greatly inhibited, even at low proportions of 293Cre cells (FIG. 5). Similarly, the presence of as few as 33% of cells expressing Cre in the mixed cultures was sufficient to prevent detection of the full size CMV-GALV vector (2.6 kb) in Hirt DNA from transfected cultures; in mixed cultures with 33% or more of Cre-expresing cells, only 0.4 kb GALV-minus vector was detected in Hirt DNA supernatants (data not shown). Therefore, Cre-mediated excision of FMP coding sequences is sufficient to stop the continued formation of syncytia.

### Example 2

### Donation in Trans of Tissue-specific Transcription Factors by Cell Fusion Activates Cre and Stops Further Syncytial Development

In order to test whether cells of one tissue type, recruited into developing syncytia formed by a heterologous tissue type, can provide transcription factors in trans to activate gene expression from an engineered promoter co-delivered with the FMG cDNA, the following experiment was performed.

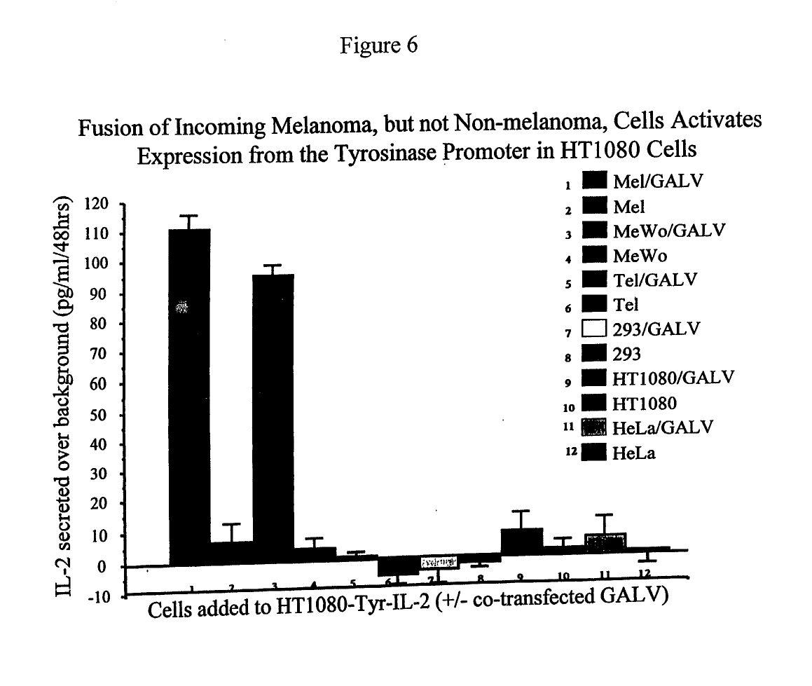

The murine tyrosinase promoter is strongly active in melanoma cells but substantially inactive in HT1080 cells. Therefore, HT1080 cells, stably transfected with the murine tyrosinase promoter directing expression of the IL-2 gene, were transfected with the CMV-GALV plasmid. The transfected cells expressed only background levels of IL-2, as detetced by ELISA. When non-melanoma cells were mixed with the HT 1 080-IL-2/GALV transfected population, IL-2 was not detected above background. In contrast, when melanoma cells (Mel624 or MeWo) cells were added to the HT 1080-IL-2/GALV transfected population, IL-2 expression was activated, presumably by donation of tyrosinase-activating transcription factors from the incoming melanoma cells (FIG. 6). Therefore, tissue-specific transcription factors donated by a fusion acceptor are sufficient to activate transcription of a gene carried by the fusion donor.

Taken together with the conclusions of the experiments in Example 1, these data provide support for the ability of tissue-specific transcription factors expressed in non-tumor fusion partners to activate expression of a recombinase, such as Cre, that then mediates the excision of loxP-flanked FMP-coding sequences and stops syncytial formation at the boundary of a tumor.

### Example 3

### Tumor/Non-tumor Fusion Studies and Tissue-specific Cre Regulation

In order to test the ability of tissue-specific transcription factors to activate expression of a tissue-specific promoter-driven Cre gene in a fusion partner and thereby halt the formation of syncytia including normal, non-tumor cells, the following will be performed.

In a first approach, murine melanoma cells (e.g., Mel624 or MeWo) are transfected with a vector carrying a LoxP-flanked FMG cassette driven by the murine tyrosinase promoter and a Cre cassette driven by the albumin promoter/enhancer (Pinkert et al., 1987, Genes Dev. 1: 268). Transfected cells are introduced to normal mice, e.g., via tail vein injection or, alternatively, via portal vein injection. Control mice are injected with melanoma cells transfected with a vector containing only the LoxP-flanked tyrosinase-driven FMG cassette. After a suitable amount of time (e.g., 5 days to 3 weeks), controls and experimental animals are killed and their livers examined for melanoma metastases and signs of syncytia formation. One looks for differences in the amount and size of metastases and differences in the involvement of surrounding hepatic tissue in syncytia between the experimental and control groups. Those animals receiving the Cre cassette in addition to the FMG cassette activate the Cre gene upon introduction of liver-specific transcription factors following fusion with hepatocytes, thereby excising and inactivating the FMG cassette, limiting ongoing syncytial formation.

Another approach is to administer metastatic murine melanoma cells to mice and allow liver tumors to form. A retroviral vector capable of infecting melanoma cells and comprising a LoxP-flanked FMG cassette driven by the tyrosinase promoter and an albumin enhancer/promoter-driven Cre cassette is administered by tail vein injection. Controls include animals injected with a vector containing only the LoxP-flanked FMG cassette and animals receiving no viral vector. After an appropriate period of time, animals in each group are killed and examined (i.e., by light microscopy for overall morphology, and by immunohistochemical analysis using melanoma-specific and viral-protein specific or even Cre-specific antibodies) for tumor size and number, evidence of syncytial formation, and involvement of non-tumor tissues in syncytia. It is expected that there will be fewer tumors in animals receiving an FMG cassette, relative to those receiving only melanoma cells, but that those animals receiving the albumin-driven Cre construct will exhibit less normal tissue involvement in the syncytia.

### Example 4

### RT-PCR for GALV mRNA

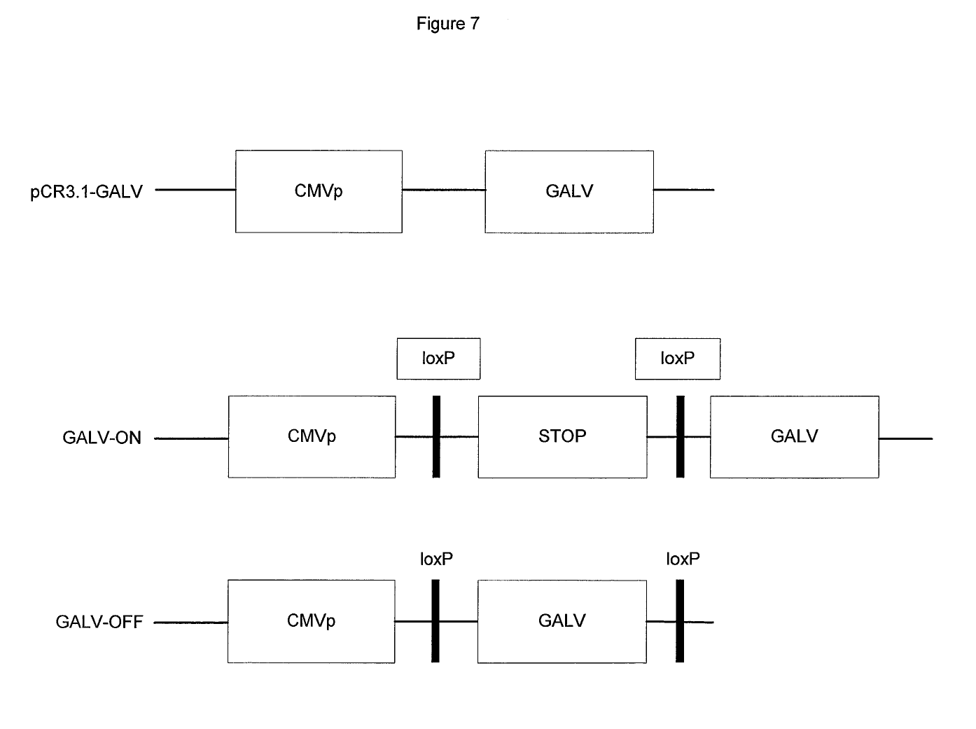

HCT-116 colorectal and 293-Cre cells were grown to 70% confluence in 25 cm2 flasks (Becton Dickinson Labware, Franklin Lakes, N.J.). The cells were transfected with one of the following constructs: pCR3.1-GALV, GALV-ON, or GALV-OFF (FIG. 7). All transfections were performed with 1 μg of plasmid DNA using the Effectene lipid reagent (Qiagen Inc., Valencia, Calif.) according to the manufacturer's instructions. Cells were harvested at 18 hours and 24 hours, and RNA was prepared using the RNeasy Kit (Qiagen Inc.) according to the manufacturer's instructions. The RNA samples were treated with DNase I (Roche Diagnostics GmbH, Mannheim, Germany) at 37° C. for 45 minutes, followed by inactivation of the DNase by heating at 65° C. for 5 minutes. A reverse transcriptase (RT) reaction was performed with 2 μg of RNA using the First Strand cDNA Synthesis Kit (Boehringer Mannheim, Mannheim, Germany) according to the manufacturer's instructions. One microlitre of the resulting cDNA was used in a PCR reaction with the following primers: 5′-GTCCTTGTGGAACAAGGACCT-3′ and 5′-CAGCTTATGGTTGGAGGGGAGC-3′. Amplification was performed using the following conditions: 94° C. for 4 minutes, 30 cycles of 94° C. for 1 minute, 56° C. for 1 minute 30 seconds, 72° C. for 3 minutes, followed by a 7 minute extension at 72° C. Samples (20 μL) were run on a 1.5% agarose gel (BioWhittaker Molecular Applications, Rockland, Me.) containing ethidium bromide.

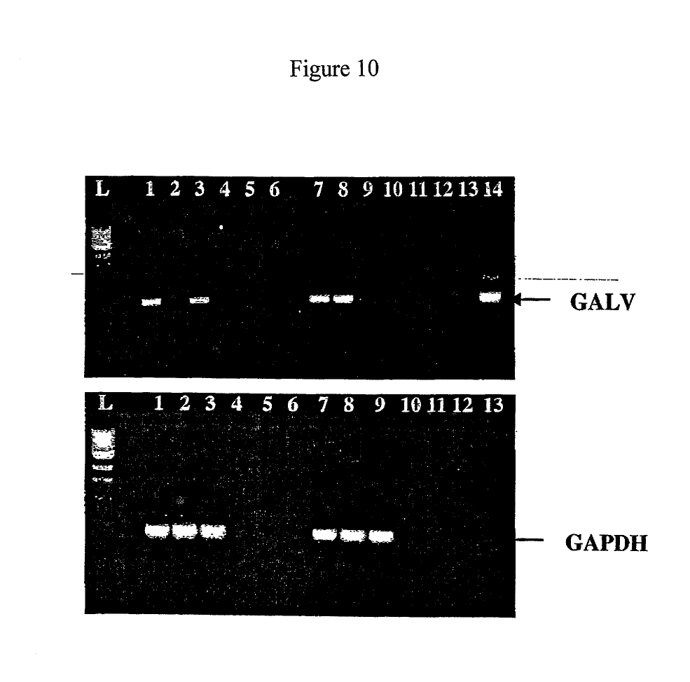

Transfection of pCR3.1-GALV and GAIV-OFF in to HCT-116 cells generated strong signals for GALV mRNA by RT-PCR (FIG. 10; Upper panel, lanes 1 and 3), whereas the GALV-ON construct gave a very weak signal (FIG. 10; Upper panel, lane 2). In contrast, transfection of pCR3.1-GALV and GALV-ON in to 293-Cre cells generated a strong signal for GALV mRNA by RT-PCR (FIG. 10; Upper panel, lanes 7 and 8), whereas the GALV-OFF construct gave a very weak signal (FIG. 10; Upper panel, lane 9). In all cases, the reverse transcriptase-negative control PCRs gave no signal for GALV (FIG. 10; lanes 4-6 and 10-12), confirming the absence of contamination of the RNA preparations with plasmid DNA. Similarly, the GAPDH controls confirmed that equal amounts of RNA had undergone the reverse traiiscriptase reaction and subsequent PCR step (FIG. 10; Lower panel, lanes 1-3 and 7-9). These data demonstrate the activity of Cre recombinase (in cells expressing the recombinase constitutively) in excising an FMG cassette flanked by loxP sites in vitro.

### Example 5

### PCR from Hirt DNA Extracts

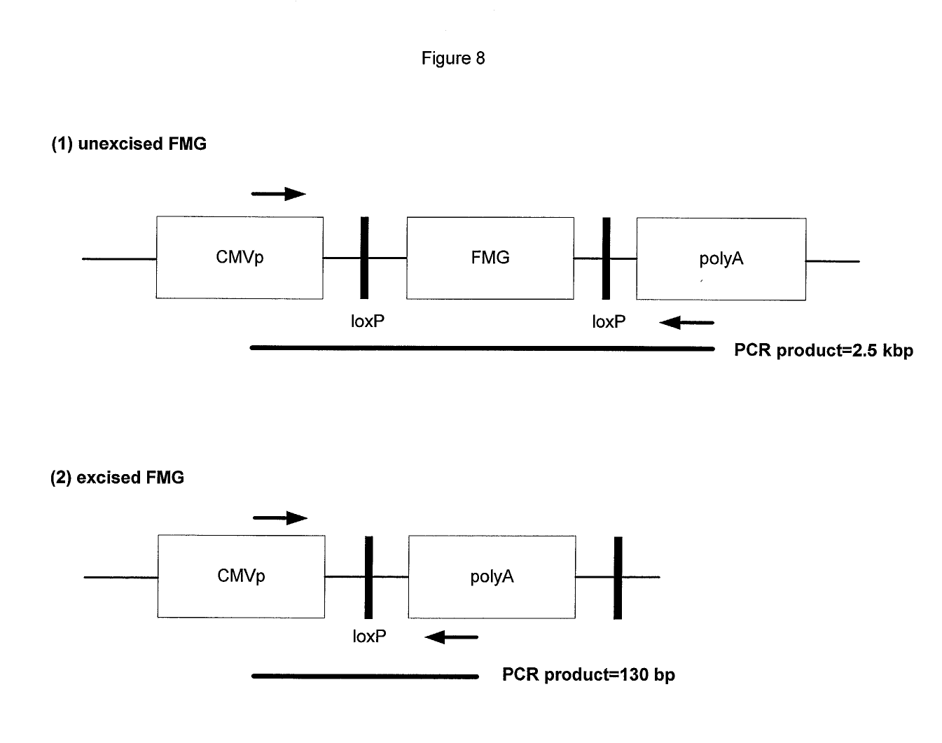

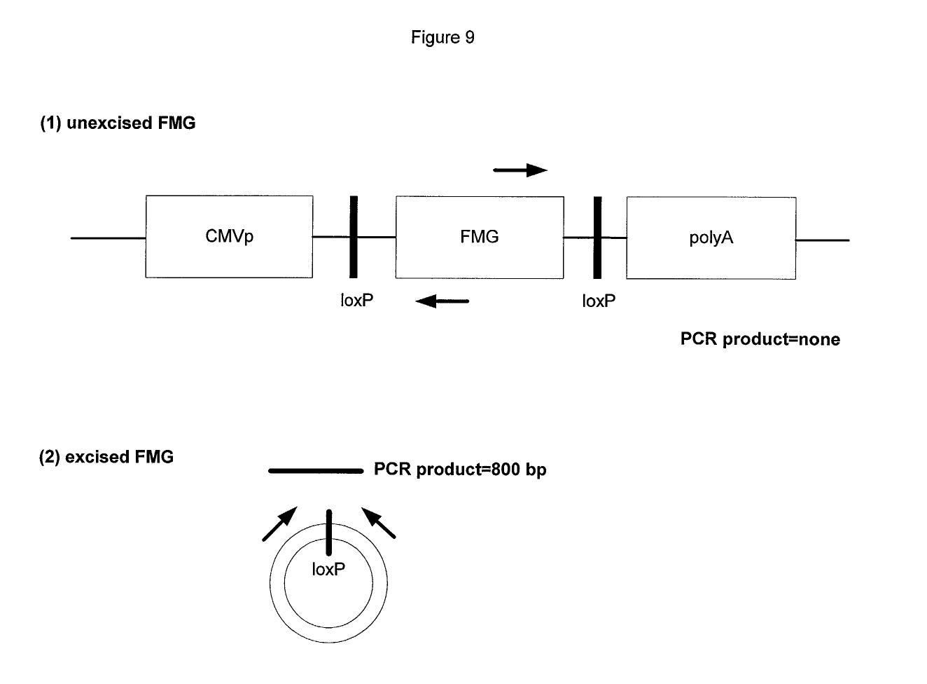

HCT-116 colorectal and 293-Cre cells were grown to 70% confluence in 25 cm² flasks (Becton Dickinson Labware). The cells were transfected with one of the following constructs: pCR3. 1-GALV, GALV-ON, or GALV-OFF. All transfections were performed with 1 μg of plasmid DNA using the Effectene lipid reagent (Qiagen Inc.) according to the manufacturer's instructions. At 24 hours, the medium was removed and the cells were washed twice with phosphate buffered saline (PBS). Hirt buffer (650 μL) was added and the flask was incubated for 10 minutes at room temperature. The cells were scraped off, collected into 1.5 mL microfuge tubes and 163 μL of 5 M sodium chloride were added. Samples were stored at −20° C. for 1 hour, thawed and centrifuged at 14000 rpm at 4° C. for 90 minutes. The supernatant was treated with 6.5 μL of a 20 mg/mL solution of Pronase (Sigma Chemical Co., St Louis, Mo.) at 37° C. for 1 hour and the samples were extracted twice with phenol/chloroform/IAA and once with chloroform. A tenth of the volume of 3 M sodium acetate (pH5.4) was added and mixed followed by 2 volumes of ice cold ethanol and the cDNA was precipitated overnight at −20° C. Subsequently, DNA was recovered by centrifugation at 14,000 rpm for 1 hour, washed with 70% ethanol, air dried and resuspended in 40 μL Tris EDTA containing 50 μg/ml RNase (Roche Diagnostics GmbH). Diagnostic PCR was performed using the two primer pairs as detailed in FIGS. 8 and 9. The first primer pair had the following sequences: 5′-CGTGTACGGTGGGAGGTCTATATA-3′ and 5′-CTCATCAATGTATCTTATCACGCG-3′. In the presence of unexcised GALV, a PCR band of 2.5 kbp was anticipated. In the event that the GALV sequence had been excised between the loxP sites, a band of 130 bp was anticipated (FIG. 8). Amplification was performed using the following conditions: 94° C. for 4 minutes, 30 cycles of 94° C. for 1 minute, 56° C. for 1 minute 30 seconds, 72° C. for 3 minutes, followed by a 7 minute extension at 72° C. The second primer pair had the following sequences: 5′-ACATAGACCACTCAGGTGCAG-3′ and 5′-TACCTGCCAAGTGAGGGTCAT-3′. In the presence of unexcised GALV, no PCR band was anticipated. In the event that the GALV sequence had been excised between the loxP sites, a band of 800 bp was anticipated (FIG. 9). Amplification was performed using the following conditions: 94° C. for 4 minutes, 30 cycles of 94° C. for 1 minute, 56° C. for 1 minute 30 seconds, 72° C. for 3 minutes, followed by a 7 minute extension at 72° C.

In subsequent studies, HCT-116 cells were transfectcd with one of the following constructs: pCR3,1-GALV, GALV-ON, or GALV-OFF. All transfections were performed with 1 μg of plasmid DNA using the Effectene lipid reagent (Qiagen Inc.) according to the manufacturers' instructions. The medium was removed at 20 hours and the cells were harvested and mixed in a ratio of 1:2 with various untransfected cells. Cells were harvested at 24 hours and 48 hours for Hirt extraction as detailed above. The same PCR primers (FIGS. 8 and 9) were used to probe for evidence of activity of the Cre/loxP switch activated by provision of Cre recombinase in trans from cells fusing into developing syncytia.

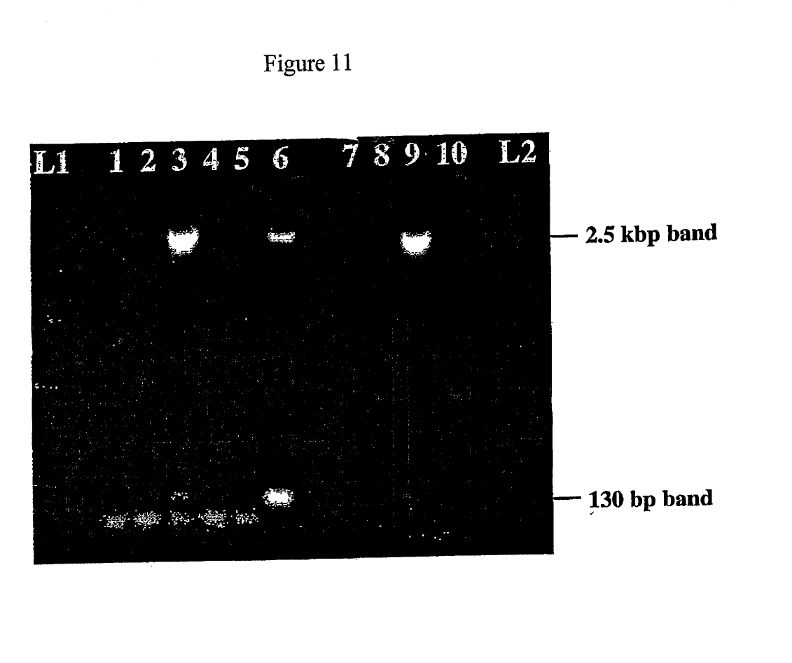

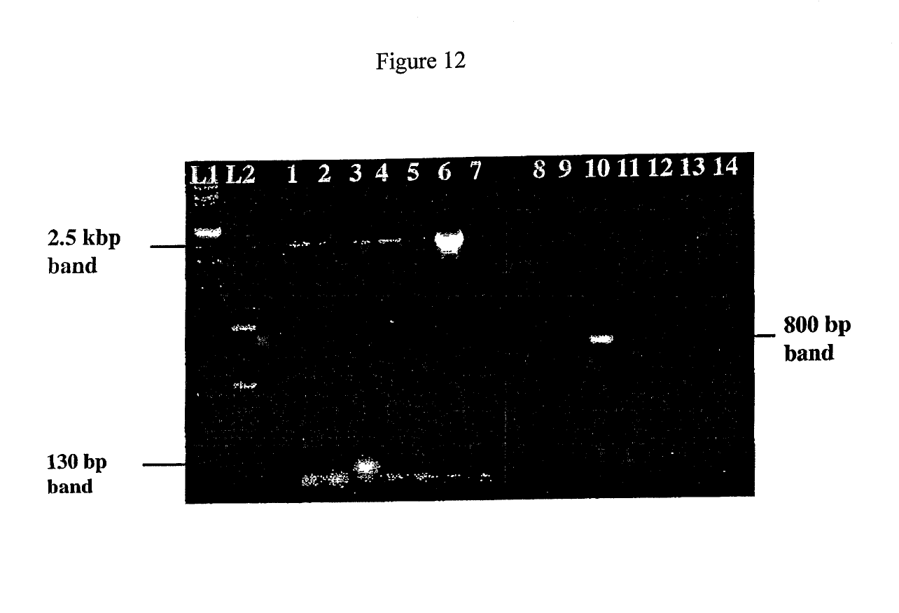

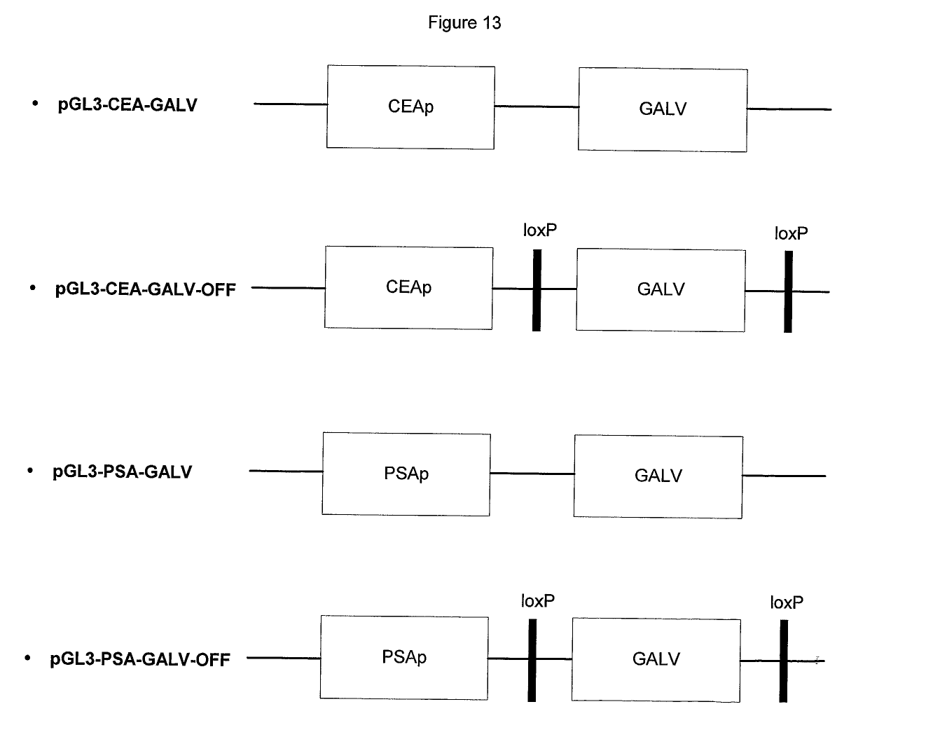

PCR using the first primer pair (FIG. 8) from the Hirt extracted DNA demonstrated the presence of the small 130 bp band following transfection of 293-Cre cells with the GALV-OFF construct (FIG. 11). There was only a very weak 2.5 kbp signal (FIG. 11; lane 6). These data are consistent with excision of the GALV sequence between the loxP sites in the GALV-OFF construct. In contrast, the full length 2.5 kbp GALV sequence was strongly present after transfection of the HCT-116 cells (FIG. 11; lane 3). There was a weak signal at 130 bp, perhaps due to some spontaneous loss of the GALV sequence in the plasmid mediated by recombination between the loxP sites (a supposition supported by the presence of this same weak band in the PCR from the cDNA positive control (FIG. 11; lane 9)). In addition, PCR using the second primer pair (FIG. 9) revealed evidence of excision of the GALV sequence by means of generation of an 800 bp PCR band only in the 293-Cre cells transfected with the GALV-OFF construct (data not shown). In the cell mixing experiment, the addition of 293-Cre cells to HCT-116 cells transfected with the GALV-OFF construct resulted in excision of the GALV sequence, as seen by the generation of the small 130 bp band (FIG. 12; lane 3) with the first primer pair (FIG. 7). Under the same conditions, the second primer pair (FIG. 2) demonstrated the formation of the excised GALV sequence (FIG. 12; lane 10).

This study demonstrates the activity of Cre recombinase in excising an FMG cassette flanked by loxP sites in vitro, in cells expressing Cre recombinase constitutively. PCR of Hirt extracted plasmid cDNA also demonstrates the ability of Cre recombinase delivered in trans into an evolving syncytium to mediate excision of the FMG cassette.

### Example 6

### The Use of Two Separate Tissue/Tumor Specific Promoters to Drive Differential Expression of a FMG Transgene and a Neutralizing cre Recombianase

FMG are viral gene products which cause cell fusion generating large syneytia. They are powerful cytoreductive agents (Bateman et al. Cancer Res. 60, 1492-7, 2000). Two separate tissue/tumor specific promoters were used to drive differential expression of a FMG transgene and a neutralizing cre recombinase (cre) gene as a model system for limiting the toxicity of FMG-based gene therapy. TelCeB6 cells (lacZ+ve) were transiently transfected with plasmids encoding FMG (Gibbon Ape Leukaemia Virus envelope (GALV) or Measles virus F and H genes) and added to untransfected homologous or heterologous tumour (HeLa. Mel624) and normal (HUVEC and HMEC) cell lines. Cell fusion extended into each cell line as shown by a mixed population of lacZ+ve and five nuclei within syncytia. These finding indicate that in vivo FMG CGT may cause cell fusion to extend beyond tumor masses into adjacent normal tissues with resulting toxicity. This indicates that the Cre-loxP system can be used to control GALV expression by generating CMV promoter-driven constructs either with GALV flanked by two loxP sites (GALV-OFF) or with GALV preceded by a transcription termination (STOP) cassette flanked by two loxP sites (GALV-ON). Transient transfection in HT-1080 cells stably expressing cre resulted in abrogation of syncytial formation for GALV-OFF and significant syncytial formation for GALV-ON. GALV-OFF TSP-driven constructs using the CEA promoter have been generated. A construct with cre under the liver-specific Albumin enhancer-promoter (pGL3-Alb-cre) has also been generated. Colorectal (HT29, HCT-116, LoVo, SW620 and SW1116) and non-colorectal (TelCeB6, HT-1080) cancer cell lines will be transiently transfected with CEA-GALV-OFF and mixed with liver (HuH7, HepG2) cell lines transfected with pGL3-Alb-cre construct. In addition, HuH7 and HepG2 cell lines stably expressing cre and a prostate specific antigen enhancer/promoter will be tested in this same system.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. A recombinant nucleic acid vector comprising a first expression cassette comprising a first promoter operably linked to a nucleic acid sequence encoding a syncytium-inducing polypeptide, wherein said first expression cassette is flanked on either side by a site recognized by a recombinase.

2. The recombinant nucleic acid vector of claim 1, further comprising a second expression cassette comprising a tissue-specific promoter operably linked to a nucleic acid sequence encoding said recombinase.

3. The recombinant nucleic acid vector of claim 1, wherein said first promoter is active in malignant cells, and wherein said tissue specific promoter is active in non-malignant cells of the same lineage as said malignant cells but is substantially inactive in said malignant cells.

4. The recombinant nucleic acid vector of claim 1, wherein said recombinase is selected from the group consisting of Cre recombinase, FLP recombinase, Gin recombinase, Pin recombinase, and lambda phage Integrase, and said site is susceptible to cleavage with said recombinase.

5. The recombinant nucleic acid vector of claim 1, wherein said first promoter is a tumor-specific promoter.

6. The recombinant nucleic acid vector of claim 5, wherein said tumor specific promoter is selected from the group consisting of a carcinoembryonic antigen promoter, an alphafetoprotein promoter, a tyrosinase promoter, an Erb-B2 promoter and a myelin basic protein promoter.

7. The recombinant nucleic acid vector of claim 1, wherein said sequence which encodes a syncytium-inducing polypeptide encodes an FMG.

8. The recombinant nucleic acid vector of claim 7, wherein said FMG is a viral FMG.

9. The recombinant nucleic acid vector of claim 8, wherein said viral FMG is selected from the group consisting of type G membrane glycoprotein of rabies virus, type G membrane glycoprotein of Mokola virus, type G membrane glycoprotein of vesicular stomatitis virus, type G membrane glycoprotein of Togaviruses, murine hepatitis virus JHM surface projection protein, porcine respiratory coronavirus spike glycoprotein, porcine respiratory coronavirus membrane glycoprotein, avian infectious bronchitis spike glycoprotein and its precursor, bovine enteric coronavirus spike protein, paramyxovirus SV5 F protein, Measles virus F protein, canine distemper virus F protein, Newcastle disease virus F protein, human parainfluenza virus 3 F protein, simian virus 41 F protein, Sendai virus F protein, human respiratory syncytial virus F protein, Measles virus hemagglutinin, simian virus 41 hemagglutinin neuraminidase proteins, human parainfluenza virus type 3 hemagglutinin neuraminidase, Newcastle disease virus hemagglutinin neuraminidase, human herpesvirus 1 gH, simian varicella virus gH, human herpesvirus gB proteins, bovine herpesvirus gB proteins, cercopithecine herpesvirus gB proteins, Friend murine leukemia virus envelope glycoprotein, Mason Pfizer monkey virus envelope glycoprotein, HIV envelpoe glycoprotein, influenza virus hemaglutinin, poxvirus membrane glycoproteins, mumps virus hemaglutinin neuraminidase, mumps virus glycoproteins F1 and F2, West Nile virus membrane glycoprotein, herpes simplex virus membrane glycoprotein, Russian Far East encephalitis virus membrane glycoprotein, Venezuelan equine encephalitis virus membrane glycoprotein and varicella virus membrane glycoprotein.

10. The recombinant nucleic acid vector of claim 1, wherein said vector is a retroviral vector.

11. A cell comprising a vector of claim 1.

12. A recombinant expression cassette system comprising a first expression cassette comprising a first promoter operably linked to a nucleic acid sequence encoding a syncytium-inducing polypeptide, wherein said first expression cassette is flanked on either side by a site recognized by a recombinase; and a second expression cassette comprising a tissue-specific promoter operably linked to a nucleic acid sequence encoding said recombinase.

13. The expression cassette system of claim 12, wherein said first and said second expression cassettes are encoded on a single vector nucleic acid.

14. The expression cassette system of claim 12, wherein said first and said second expression cassettes are encoded on separate nucleic acid vectors.

15. The expression cassette system of claim 12, wherein said first promoter is active in malignant cells, and wherein said tissue specific promoter is active in non-malignant cells of the same lineage as said malignant cells but is substantially inactive in said malignant cells.

16. The expression cassette system of claim 12, wherein said recombinase is selected from the group consisting of Cre recombinase, FLP recombinase, Gin recombinase, Pin recombinase, and lambda phage Integrase, and said site is susceptible to cleavage with said recombinase.

17. The expression cassette system of claim 12, wherein said first promoter is a tumor specific promoter.

18. The expression cassette system of claim 17, wherein said tumor specific promoter is selected from the group consisting of a carcinoembryonic antigen promoter, an alphafetoprotein promoter, a tyrosinase promoter, an Erb-B2 promoter and a myelin basic protein promoter.

19. The expression cassette system of claim 12, wherein said sequence which encodes a syncytium-inducing polypeptide encodes an FMG.

20. The expression cassette system of claim 19, wherein said FMG is a viral FMG.

21. The expression cassette system of claim 20 wherein said viral FMG is selected from the group consisting of type G membrane glycoprotein of rabies virus, type G membrane glycoprotein of Mokola virus, type G membrane glycoprotein of vesicular stomatitis virus, type G membrane glycoprotein of Togaviruses, murine hepatitis virus JHM surface projection protein, porcine respiratory coronavirus spike glycoprotein, porcine respiratory coronavirus membrane glycoprotein, avian infectious bronchitis spike glycoprotein and its precursor, bovine enteric coronavirus spike protein, paramyxovirus SV5 F protein, Measles virus F protein, canine distemper virus F protein, Newcastle disease virus F protein, human parainfluenza virus 3 F protein, simian virus 41 F protein, Sendai virus F protein, human respiratory syncytial virus F protein, Measles virus hemagglutinin, simian virus 41 hemagglutinin neuraminidase proteins, human parainfluenza virus type 3 hemagglutinin neuraminidase, Newcastle disease virus hemagglutinin neuraminidase, human herpesvirus 1 gH, simian varicella virus gH, human herpesvirus gB proteins, bovine herpesvirus gB proteins, cercopithecine herpesvirus gB proteins, Friend murine leukemia virus envelope glycoprotein, Mason Pfizer monkey virus envelope glycoprotein, HIV envelope glycoprotein, influenza virus hemaglutinin, poxvirus membrane glycoproteins, mumps virus hemaglutinin neuraminidase, mumps virus glycoproteins F1 and F2, West Nile virus membrane glycoprotein, herpes simplex virus membrane glycoprotein, Russian Far East encephalitis virus membrane glycoprotein, Venezuelan equine encephalitis virus membrane glycoprotein and varicella virus membrane glycoprotein.

22. The expression cassette system of any one of claims 12, wherein said expression cassette system is encoded one or more retroviral vectors.

23. A cell comprising the expression cassette system of claims 12.

24. A therapeutic composition comprising a cell of any one of claims 1 or 12 in admixture with a physiologically acceptable carrier.

25. A method of reducing tumor size, said method comprising the step of:
(a) permitting expression in an individual in need of treatment for a disease caused by malignant cells of a first expression cassette comprising a tumor specific promoter operably linked to a nucleic acid sequence encoding a syncytium-inducing polypeptide, wherein said first expression cassette is flanked on either side by a site recognized by a recombinase; and
(b) a second expression cassette comprising a tissue-specific promoter operably linked to a nucleic acid sequence encoding said recombinase, wherein said tumor-specific promoter is active in said malignant cells, and said tissue specific promoter is active in non-malignant cells of the same lineage as the malignant cells, but substantially inactive in said malignant cells, wherein said expression results in a reduction in tumor size.

26. The method of claim 25, wherein said step of permitting expression comprises the step of administering first and second expression cassettes to an individual in need of treatment for a disease caused by malignant cells.

27. The method of claim 25, wherein said recombinase is cre recombinase and said site recognized by a recombinase is a loxp site.

28. The method of claim 25, wherein said tumor-specific promoter is selected from the group consisting of a carcino embryonic antigen promoter, an alphafetoprotein promoter, a tyrosinase promoter, an Erb-B2 promoter and a myelin basic protein promoter.

29. The method of claim 28, wherein said tumor specific promoter is the carcinoembryonic antigen promoter.

30. The method of claim 25, wherein said sequence which encodes a syncytium-inducing polypeptide encodes an FMG.

31. The method of claim 30, wherein FMG is a viral FMG.

32. The method of claim 31, wherein said viral FMG is selected from the group consisting of type G membrane glycoprotein of rabies virus, type G membrane glycoprotein of Mokola virus, type G membrane glycoprotein of vesicular stomatitis virus, type G membrane glycoprotein of Togaviruses, murine hepatitis virus JHM surface projection protein, porcine respiratory coronavirus spike glycoprotein, porcine respiratory coronavirus membrane glycoprotein, avian infectious bronchitis spike glycoprotein and its precursor, bovine enteric coronavirus spike protein, paramyxovirus SV5 F protein, Measles virus F protein, canine distemper virus F protein, Newcastle disease virus F protein, human parainfluenza virus 3 F protein, simian virus 41 F protein, Sendai virus F protein, human respiratory syncytial virus F protein, Measles virus hemagglutinin, simian virus 41 hemagglutinin neuraminidase proteins, human parainfluenza virus type 3 hemagglutinin neuraminidase, Newcastle disease virus hemagglutinin neuraminidase, human herpesvirus 1 gH, simian varicella virus gH, human herpesvirus gB proteins, bovine herpesvirus gB proteins, cercopithecine herpesvirus gB proteins, Friend murine leukemia virus envelope glycoprotein, Mason Pfizer monkey virus envelope glycoprotein, HIV envelpoe glycoprotein, influenza virus hemaglutinin, poxvirus membrane glycoproteins, mumps virus hemaglutinin neuraminidase, mumps virus glycoproteins F1 and F2, West Nile virus membrane glycoprotein, herpes simplex virus membrane glycoprotein, Russian Far East encephalitis virus membrane glycoprotein, Venezuelan equine encephalitis virus membrane glycoprotein and varicella virus membrane glycoprotein.

33. The method of claim 25, wherein said step of administering comprises administering one or more retroviral vectors comprising said first and second expression cassettes.

34. The method of claim 25, wherein said step of administering comprises administering a cell comprising said one or more recombinant nucleic acid vector.

35. An expression cassette system comprising:
(a) a first expression cassette comprising an hypoxic response element (HRE) operably linked to a nucleic acid sequence encoding a syncytium-inducing polypeptide, wherein said nucleic acid sequence encoding a syncytium-inducing polypeptide is flanked on either side by a sequence recognized by a recombinase;
(b) a second expression cassette comprising a tumor specific promoter operably linked to a nucleic acid sequence encoding a cytotoxic gene product; and
(c) a third expression cassette comprising a tumor specific promoter operably linked to said nucleic acid sequence encoding said recombinase.

36. An expression cassette system comprising:
(a) a first expression cassette comprising an hypoxic response element (HRE) operably linked to a nucleic acid sequence encoding a syncytium-inducing polypeptide, wherein said nucleic acid sequence encoding a syncytium-inducing polypeptide is flanked on either side by sequences recognized by a recombinase;
(b) a second expression cassette comprising a tumor specific promoter operably linked to a nucleic acid sequence encoding a cytokine; and
(c) a third expression cassette comprising a tumor specific promoter operably linked to said nucleic acid sequence encoding said recombinase.

37. The expression cassette system of claim 35 or 36, wherein said vector is a retroviral vector.

38. The expression cassette system of claim 35 or 36, wherein said tumor specific promoter is selected from the group consiting of a carcinoembryonic antigen promoter, an alphafetoprotein promoter, a tyrosinase promoter, an Erb-B2 promoter and a myelin basic protein promoter.

39. The expression cassette system of claim 38, wherein said tumor specific promoter is a carcinoembryonic antigen promoter.

40. The expression cassette system of claim 35, wherein said cytotoxic gene product is selected from the group consisting of HSV thymidine kinase, cytosine deaminase, nitroreductase, and a viral FMG.

41. The expression cassette system of claim 36, wherein said cytokine is selected from the group consisting of IL-1, IL-2, IL-3, IL-4, IL-5, IL-6, IL-7, IL-12, GM-CSF, IFN-γ and TNF-α.

42. A cell comprising an expression cassette system of claim 35 or 36.

43. The cell of claim 42, wherein said cell is a macrophage.

44. A method of reducing the size of a tumor in an individual said method comprising the step of permitting the expression in an individual of an expression cassette system comprising:
(a) a first expression cassette comprising a nucleic acid sequence encoding a syncytium-inducing polypeptide, operably linked to an hypoxic response element (HRE), wherein said nucleic acid sequence encoding a syncytium-inducing polypeptide is flanked on either side by sequences recognized by a recombinase,
(b) a second expression cassette comprising a nucleic acid sequence encoding a cytotoxic gene product, operably linked to a tumor specific promoter, and
(c) a third expression cassette comprising a nucleic acid sequence encoding said recombinase, operably linked to said tumor specific promoter, wherein expression of said expression cassette system reduces the size of a tumor.

45. The method of claim 46, wherein said step of permitting expression comprises introducing said expression cassette system to a macrophage and introducing said macrophage to said individual.

46. A method of reducing the size of a tumor in an individual said method comprising the step of permitting the expression in an individual of an expression cassette system comprising:
(a) a first expression cassette comprising a nucleic acid sequence encoding a syncytium-inducing polypeptide, operably linked to an hypoxic response element (HRE), wherein said nucleic acid sequence encoding a syncytium-inducing polypeptide is flanked on either side by sequences recognized by a recombinase,
(b) a second expression cassette comprising a nucleic acid sequence encoding a cytokine, operably linked to a tumor specific promoter, and
(c) a third expression cassette comprising a nucleic acid sequence encoding said recombinase, operably linked to said tumor specific promoter, wherein expression of said expression cassette system reduces the size of a tumor.

47. The method of claim 46, wherein said step of permitting expression comprises introducing said expression cassette system to a macrophage and introducing said macrophage to said individual.

48. A macrophage-tumor cell hybrid.

49. The macrophage-tumor cell hybrid of claim 48, wherein said hybrid comprises an expression cassette system of claims 35.

50. The macrophage-tumor cell hybrid of claim 48, wherein said hybrid comprises an expression cassette system of claims 36.

51. A cell-tumor cell hybrid, wherein said hybrid comprises a hypoxic transcription factor.

52. The cell-tumor cell hybrid of claim 51, wherein said hybrid an expression cassette system of claim 35.

53. The cell-tumor cell hybrid of claim 51, wherein said hybrid an expression cassette system of claim 36.
